(19) United States
(12) Patent Application Publication
(10) Pub. No.: US 2025/0049366 A1
(43) Pub. Date: Feb. 13, 2025

(54) AUTOMATIC URINE MEASURING DEVICE

(71) Applicant: JUNGLE VENTURES SL, Barcelona (ES)

(72) Inventors: Josep MASÓ, Barcelona (ES); Pär BLANKING, Barcelona (ES); Jordi FERRÉ, Barcelona (ES); Sergi CÓNSUL, Barcelona (ES); Daniel VEA, Barcelona (ES); Adrià ARNASTE, Barcelona (ES)

(21) Appl. No.: 18/719,192

(22) PCT Filed: Dec. 13, 2022
(86) PCT No.: PCT/ES2022/070796

§ 371 Date: Jun. 12, 2024

(30) Foreign Application Priority Data
Dec. 13, 2021 (ES) U202132437

(51) Int. Cl. A61B 5/20; A61B 5/00; G16H 40/63

(52) U.S. Cl. CPC A61B 5/208; A61B 5/6891; G16H 40/63

(57) ABSTRACT

The invention relates to a urinary measuring device for taking and measuring urine samples, configured to be arranged in a urinary receptacle. The device is characterized in that it comprises: a) a funnel for sample collection, comprising a main opening; b) a urine collection unit connected to the opening for receiving the sample from the funnel; and c) a casing for electronics positioned close to the urine collection unit, the casing for electronics containing a light emitter arranged for emitting light on the urine collection unit and a sensor arranged for measuring the light received from the urine collection unit.

### OBJECT OF THE INVENTION

The present invention belongs to the field of urinary analysis devices, and particularly to a measuring device for taking urine samples, measuring several parameters of the sample, and recording the measurements, configured to be arranged in a urinary receptacle. The invention also relates to the use of the urinary measuring device for urine analysis and to a method for urine analysis, as well as the implementation thereof by a computer.

### BACKGROUND OF THE INVENTION

Biometric parameter analysis and monitoring is a fundamental aspect for the early detection of different health problems, as well as for the performance of continuous follow-ups. Blood analysis stands out among the most commonly used reference methods. Although blood extractions can be performed for subsequent blood measurement, they are invasive, require the intervention of a professional, and are not practical for frequent implementation. In fact, waste from blood and excess blood are removed through urine, serving in turn as means for regulating the balances of different components in the body, such as electrolytes and water. Urine analysis or urinalysis is therefore a convenient way to detect and control different diseases by means of detecting solutes therein. Given that a large amount of molecules, such as glucose, hormones, and derivatives from different biological processes are discharged from the body through urine, urinalysis is a highly effective, non-invasive way to monitor the condition of a subject in multiple aspects, such as hydration, creatine, or glucose levels. Urinalyses can be performed by measuring the amounts of different solutes in urine, analyzing physical properties thereof, such as color, smell, taste, viscosity, conductivity, and pH, among others.

However, current urinalyses are based on either colorimetric analysis, requiring the use of and continuous spending on consumables, or on the use of complex laboratory analysis methods such as mass spectrometry, involving expensive equipment. In this sense, in order to perform analyses, patients are usually required to travel to a center where tests will be performed on said patients, to urinate into a container, and to deliver the sample to a technician for subsequent analysis. This involves a series of drawbacks for the patients who have to travel and provide urine samples to a third party, leading to patient discouragement and a lack of recurrence in analyses. Moreover, laboratory technicians are the ones who carry out the tests, requiring the handling of biological samples and the use of advanced instrumentation, involving a cost in terms of time as well as human and technical resources.

There is therefore a need for a urine measuring system that is as minimally invasive as possible for the patient, as simple as possible for the technician, or can be used without the intervention of a technician, with a real-time measurement and a low cost so that it can be implemented on a recurring basis.

### SUMMARY OF THE INVENTION

A first aspect of the invention relates to a urinary measuring device for taking and measuring urine samples, configured to be arranged in a urinary receptacle; characterized in that it comprises:
a. A funnel for sample collection, comprising a main opening;
b. A urine collection unit connected to the opening for receiving the sample from the funnel;
c. A casing for electronics positioned close to the urine collection unit, the casing for electronics containing a light emitter arranged for emitting light on the urine collection unit and a light sensor arranged for measuring the light received from the urine collection unit.

In a preferred embodiment, the funnel comprises a filter that at least partially covers the opening configured to prevent direct urination through the opening.

In another preferred embodiment, the funnel further comprises a gasket in a part of the urinary measuring device, the gasket configured to contact the urinary receptacle for fluidically sealing a space between the urinary measuring device and the urinary receptacle.

In another preferred embodiment, the funnel further comprises an overflow opening configured to inhibit the sample from overflowing from the funnel, and a conduit configured to receive fluid from the overflow opening, with the conduit bypassing the urine collection unit. In an even more preferred embodiment, the funnel further comprises a lid covering the overflow opening configured to prevent the sample from flowing directly through the overflow opening.

In another preferred embodiment, the light emitter and the light sensor are located in the casing for electronics for emitting and receiving light with respect to the urine collection unit at an angle comprised between 120° and 180°.

In another preferred embodiment, the casing for electronics comprises a main body, a cover, and a gasket configured to fluidically seal the interface between the main body and the cover.

In another preferred embodiment, the casing for electronics comprises a transparent protector arranged for protecting the light emitter and the light sensor against the sample splashing from the urine collection unit.

In another preferred embodiment, the urine collection unit comprises a surface arranged for maximizing the reflection of an incident light and which in turn comprises a material selected from one of the materials from the following list: ceramic, crystal, glass, porcelain, metal, silicone, wood, stones such as marble, polymers such as POM or photosensitive resin, as well as a derivative of these materials.

In another more preferred embodiment, the urinary measuring device further comprises:
d. A container for collecting fluid overflowing from the urine collection unit, comprising an outlet opening; and
e. A plurality of electrodes extending inside the container by means of arms.

The device may comprise a voltage source connected between the electrodes configured to apply a voltage signal between the electrodes and a current meter configured to measure the current between the electrodes resulting from a voltage signal applied by the voltage source. The device may comprise an impedance meter connected to the electrodes, the impedance meter configured to measure the impedance of the fluid present in the container between the electrodes.

In a preferred embodiment, the outlet opening of the container is configured to allow an outflow smaller than that of the main opening of the funnel. In another preferred embodiment, the container further comprises a side outlet opening, arranged at a greater height than that of the outlet opening and than that of the plurality of electrodes.

In a preferred embodiment, electric cables extend from the arms to the casing for electronics through one or more gaskets, in order to form an electrical connection between the electrodes and the electronics in the casing for electronics.

In a preferred embodiment, the funnel comprises a handle configured to facilitate the extraction of the urinary measuring device by means of an extraction device.

In a preferred embodiment, the casing for electronics further comprises a controller configured to control the light emitter, the light sensor, and optionally the impedance meter, and the controller is made up of a processor configured to process information from the light emitter, light sensor, and optionally from the impedance meter.

In a preferred embodiment, the urinary measuring device further comprises a valve configured to control the outflow of water from a water source to the urinary receptacle, and the device is configured to control the valve. In an even more preferred embodiment, the urinary measuring device is configured to open the valve when it detects that sample measurement has ended.

In a preferred embodiment, the urinary measuring device further comprises a user interface configured to receive identification or anonymous information of a user and to receive information about the urinary sample of the user and to show information about the condition of the sample to the user.

A second aspect of the invention relates to the use of a urinary measuring device according to any of the embodiments of the first aspect of the invention for urine analysis. In a preferred embodiment, the analysis comprises a spectrophotometric measurement. In another preferred embodiment in which the urinary measuring device comprises a plurality of electrodes and an impedance meter, the analysis comprises measuring the electrical impedance between the electrodes.

A third aspect of the invention relates to a method for urine analysis comprising the following steps:
a) Receiving a urine sample in a container directly from a user, with the container being positioned in a urinary receptacle;
b) Emitting a light on the sample by means of a light emitter;
c) Receiving the light coming from the sample by means of a light sensor;
d) Transmitting data indicative of the light received by the sample to a processor configured to determine the presence of one or more substances in the sample based on the light received by the sample; and
e) Receiving from the processor an indication of the presence of one or more substances in the sample.

In a preferred embodiment, the step of transmitting data indicative of the light received comprises the steps of calculating light absorption or reflection by the sample based on the light received and transmitting data indicative of light absorption or reflection to the processor which is configured to determine the presence of one or more substances in the sample based on light absorption or reflection by the sample.

In a preferred embodiment, the emission of a light on the sample and the reception of the light coming from the sample start when the presence of a sample in the container is detected; and the emission of light on the sample and the reception of light are stopped when the absence of a sample in the container is detected.

In a more preferred embodiment, the presence and absence of the sample is detected by means of analyzing the electric current between a plurality of electrodes resulting from a voltage signal between a plurality of electrodes, wherein a current value above a threshold value between the electrodes indicates the presence of the sample, and a current value below a threshold value between the electrodes indicates the absence of the sample. In another embodiment, the presence and absence of the sample is detected by means of analyzing the electrical impedance measured between a plurality of electrodes, wherein an impedance magnitude value below a threshold value between the electrodes indicates the presence of the sample, and/or an impedance magnitude value above a threshold value between the electrodes indicates the absence of the sample.

In a preferred embodiment, after detecting the presence of a sample in the container, the method comprises the following steps:
a) Measuring the impedance between the electrodes continuously until the absence of a sample in the container is detected;
b) Transmitting data indicative of the impedance of the sample to a processor configured to determine the concentration of the urine sample based on the measured impedance; and
c) Receiving from the processor an indication of the concentration of the sample.

The measured impedance can be a complex impedance measurement or resistance measurement.

In another preferred embodiment, after detecting the presence of a sample in the container, the method comprises the following steps:
a) Starting a stopwatch when the presence of the sample in the container is detected;
b) Stopping the stopwatch when the absence of the sample in the container is detected;
c) Calculating the time elapsed in the stopwatch;
d) Transmitting data indicative of the time elapsed to a processor configured to determine the volume of the urine sample based on the time elapsed; and
e) Receiving from the processor an indication of the volume of the sample.

In another preferred embodiment, one or more of the following characteristics of the sample are determined by means of analyzing the measured associated values of the sample in an algorithm performed by an external computer: urine density (mass per unit of volume), urine color, total urine volume, total urination time, mean urinary flow (volumetric flow rate), the presence and/or concentration of one or more substances in the urine, for example, electrolytes (for example, sodium (Na⁺), potassium (K⁺), calcium (Ca²⁺), magnesium (Mg²⁺), chloride (Cl⁻), hydrogen phosphate (HPO₄²⁻) and/or hydrogen carbonate (HCO₃⁻)), urea, creatinine, glucose, nitrates, ketones, bilirubin, proteinuria or hematuria, as well as the possibility of the user having one or more pathologies, for example dehydration, urinary tract infection, or kidney stones (urolithiasis).

In another preferred embodiment, before receiving a urine sample, the user is identified through a user interface. User identification can be performed by means of one or more of:
the identification of an RFID or NFC tag of the user which is detected by an RFID or NFC sensor of the user interface;
the input of a username and a password through a guided user interface;
the scanning of a QR code, shown on a display device of the user interface, through a software application in the mobile device of the user;
facial recognition of the user's iris or retina by means of a suitable recognition device of the user interface (for example, an eye scanner);
recognition of the user's weight and/or height by means of a scale and/or a stadiometer;
fingerprint recognition through a biometric print recognition device;
recognition of the user's voice by means of the audio recorded through a microphone of the user interface.

In some embodiments, the device is configured to measure the sample without identifying the user.

In another preferred embodiment, the method further comprises the following steps:
a) Discharging water from a water source when the absence of the sample in the container is detected;
b) Monitoring the presence of water in the container by means of the electrodes, by means of analyzing the electric current between the electrodes resulting from a voltage signal between a plurality of electrodes or by means of analyzing the electrical impedance measured between a plurality of electrodes; and
c) Determining that a new urine sample can be received when the presence of water is no longer detected by means of the electrodes, wherein a current value below a threshold value between the electrodes indicates the absence of water or an impedance magnitude value above a threshold value between the electrodes indicates the absence of water.

A fourth aspect of the invention relates to a computer program, connected with a container positioned in a urinary receptacle, comprising computer-readable instructions which, when being executed by a processor, cause the processor to execute a method according to the following steps:
a) Emitting a light by means of a light emitter on a urine sample from a user contained in the container;
b) Receiving the light coming from the sample by means of a light sensor;
c) Transmitting data indicative of the light received by the sample to a processor configured to determine the presence of one or more substances in the sample based on the light received by the sample; and
d) Receiving from the processor an indication of the presence of one or more substances in the sample.

### BRIEF DESCRIPTION OF THE FIGURES

To allow a better understanding of the present disclosure and to show how it can be carried out, reference is now made, only by way of example, to the attached schematic drawings in which:

FIG. 1 shows a perspective view (A) and a profile view with a cross-section (B) of the urinary measuring device according to one or more embodiments.

FIG. 2 shows a filter in a profile view (A), in a perspective view (B), and being placed in a funnel (C) according to one or more embodiments.

FIG. 3 shows an exploded view of the gasket and the funnel (A) and a plan view of a gasket (B) according to one or more embodiments.

FIG. 4 show a profile view (A), a plan view (B) of a funnel according to one or more embodiments. Furthermore, it shows a cross-section view (C) of the placement of the funnel with respect to the container according to one or more embodiments.

FIG. 5 shows a plan view (A) and a perspective view (B) of a funnel according to one or more embodiments.

FIG. 6 shows a plan view (A) and a profile view (B) of an optical device according to one or more embodiments. Furthermore, it shows a profile view (C) of the placement of the optical device in a casing for electronics for storing the optical system according to one or more embodiments.

FIG. 7 shows a perspective view (A) and a profile view (B) of a urine collection unit according to one or more embodiments. Furthermore, it shows a profile view (C) of the placement of the urine collection unit with respect to a casing for electronics and another profile view (C) of the interaction of light on the surface of the urine collection unit according to one or more embodiments.

FIG. 8 shows a profile view (A), a plan view (B), and a perspective view (C) of a container according to one or more embodiments.

FIG. 9 shows two embodiments (A and B) of a plan view of a handle according to one or more embodiments.

FIG. 10 shows an exploded view of a urinary measuring device according to one or more embodiments.

FIG. 11 shows a diagram of the relationship of a controller with a solenoid valve and a user interface according to one or more embodiments.

FIG. 12 shows a diagram of the urinary analysis method according to one or more embodiments.

FIG. 13 shows a diagram of an impedance meter according to one or more embodiments.

FIG. 14 shows a diagram of a computer program product according to one or more embodiments.

FIG. 15 shows a profile view of several optical systems together with their urine collection unit (A, C, and E) together with their respective standard curves (B, D, and F) with respect to a color standard according to one or more embodiments.

### DESCRIPTION OF THE INVENTION

### Definitions

The term “urinary receptacle” refers to any sanitary apparatus adapted to receive and evacuate body waste. In the present invention, a urinary receptacle can be an upright urinal, a wall urinal, a floor urinal, or a urinal adapted for women and/or men, as well as a toilet. A urinary receptacle is unlike a sample holder, such as a bottle, which receives the sample but does not dispose of it such that, after use, the user must discard the sample himself or herself.

The term “funnel” refers to an instrument which allow fluids to pass through a narrower opening from a wide opening. In some embodiments, the funnel allows all the urine sample or water deposited in any part of the funnel to be channeled to a narrower opening through which it is to flow.

The term “main opening” refers to the opening which serves as the narrower opening of a funnel.

The term “urine collection unit” refers to a container for fluid samples which allows containing a fluid sample. In the present invention, the sample is preferably contained such that it can be analyzed by means of a spectroscopy system such as the disclosed optical system. The abbreviated term “UCU” can be used alternatively.

The term “casing” refers to a system formed by at least one part forming a space therein, such that one or more elements can be stored therein. Therefore, the casing for electronics refers to a casing intended mainly for housing electronic elements therein, without being limited to any type of electronic element.

The term “light emitter” refers to an element configured to emit an electromagnetic wave, preferably being a visible or near-visible light (infrared or ultraviolet) emitter.

The term “light sensor” refers to an element configured to measure incident electromagnetic radiation and provide a response indicative of the measured electromagnetic radiation. Preferably, it is a visible or near-visible light (infrared or ultraviolet) sensor.

The term “filter” refers to an element which allows the passage of fluids therethrough, while preventing the passage of unwanted materials such as solids and/or urine bubbles.

The term “gasket” refers to an element configured to prevent or inhibit fluid communication between two spaces.

The term “lid” refers to an element configured to prevent one element from directly accessing (in a straight line) another target element. When the lid covers an opening, the lid is anything that, from a specific position, prevents another element from directly accessing the opening.

The term “protector” refers to an element configured to limit the exposure of another element to an agent. In the case of an optical system, it refers to anything that prevents agents such as lime, dirt, water, or acid, among others, from accessing the optical system. It can therefore be a chemical or mechanical substance and a liquid or solid.

The term “container” refers to an element configured to store other elements therein.

The term “handle” refers to a part of an element which can be gripped by the user or a tool for handling the element.

The term “solenoid valve” refers to an electrically or electronically actuated device for controlling the passage of a fluid therethrough.

The term “software” refers to a computer program or a set of computer programs.

The term “hardware” refers to the set of physical elements comprised in an electronic device.

The term “impedance” refers to the opposition to alternating current caused by the combined effect of resistance and/or reactance in a circuit. It will be seen that the devices and methods disclosed herein can be configured to measure admittance instead of impedance and to use equivalent threshold admittance values or machine learning algorithms trained in equivalent admittance data.

### DESCRIPTION

The present invention solves the problems set forth above by means of a urinary measuring device placed in a urinary receptacle, which is capable of taking samples, the user analyzing them in-situ and in real time, in an objective, automatic, and self-operated manner, providing measurements rapidly and being able to be reused repeatedly without the use of consumables and without the intervention of a third party at a low cost.

In-situ real-time measurements can be achieved as a result of one or more sensors present in the urinary measuring device, which allow the device to analyze different properties of a urine sample.

Due to the configuration of the device, the sample is taken and arranged facing an optical system automatically, so the sample can be measured without having to handle same.

Furthermore, through different automatic calibration and cleaning methods, one and the same device can be used by different users without requiring the intervention of a third party, since the device automatically detects the end of each urination, and after analyzing the sample, is capable of eliminating it and calibrating the device for the next user. Furthermore, the typology and configuration of the sensors and the urinary measuring system do not require the constant use of consumables for performing multiple urine analyses either.

A first aspect of the invention relates to a urinary measuring device for taking and measuring urine samples, configured to be arranged in a urinary receptacle; characterized in that it comprises:
a. A funnel for sample collection, comprising a main opening;
b. A urine collection unit (UCU) connected to the opening for receiving the sample from the funnel;
c. A casing for electronics positioned close to the UCU, the casing for electronics containing a light emitter arranged for emitting light on the UCU and a light sensor arranged for measuring the light received from the UCU.

It should be highlighted that the urinary measuring device is configured to be arranged in a urinary receptacle, and therefore, the shape of the device as a whole is configured to be adapted to urinary receptacles of a certain type. However, this should not be considered a limitation thereof, since different shapes of the device described in the present invention can be adapted to different urinary receptacles, such as a toilet or a wall urinal. Urinary receptacle should be understood to mean any type of system which allows collecting urine from a subject for treatment. Therefore, the device of the first aspect of the invention, as well as all the embodiments thereof, are configured to be adapted to upright urinals, wall urinals, ground urinals, urinals adapted for women, as well as toilets. Therefore, all the drawings herein present should be understood as a mere exemplification of the device and not as a limiting feature thereof.

It should be highlighted that the funnel comprises the main opening regardless of the placement of the main opening. It is therefore understood that the main opening can be placed at any point of the funnel. Although the funnels preferably comprise the main opening in the lower part thereof, it is not an essential feature of the present invention. Furthermore, the funnel is configured to pass the sample through the main opening. Although most funnels use ramps to direct fluid samples to the main opening by gravity, some embodiments may use other mechanisms that are capable of directing the samples, such as fluid pumps. Therefore, the funnel can be made up of any element which allows the fluid received in the funnel to be directed preferably to the main opening.

The light emitter and the light sensor form an optical system. The optical system allows emitting light in a range of wavelengths with the light emitter and detecting a frequency spectrum of the light with the light sensor. Therefore, the optical system is capable of emitting light on a sample and detecting the absorption of the light emitted by said sample, i.e., the optical system is capable of taking spectrophotometric measurements. Absorption spectrophotometry is based on the Beer-Lambert law which links the amount of light absorbed by a body with the concentration of said body in a solution and can be detected in the light transmitted through the sample or in the light reflected by the surface of the sample. The following is established for a liquid:

$$\frac{{I}_{1}}{{I}_{0}} = {10}^{- ε lc} = {10}^{A}$$

where I₀ is the incoming light intensity, I₁ the outgoing light intensity, ε the absorptivity of the medium, I the distance traveled by the aqueous medium, c the molar concentration of the body in the solution, and A the absorbance. Therefore, the absorbance of the solution with a wavelength λ can be obtained such that:

$${A}_{λ} = - {log}_{10} ( \frac{{I}_{1}}{{I}_{0}} ) .$$

Given a known device for which the distance I traveled by the light and the absorptivity of the body ε are known, the concentration of the body in the medium can be obtained such that:

$$c = \frac{A}{l ε} .$$

However, to that end, the sample must be arranged such that it is placed between the light emitter and the light sensor. In this sense, the UCU works as a container which allows storing fluid samples deposited in the device and presenting them to the optical system so as to enable the spectrophotometric analysis thereof. It should be noted that the sample can be presented to the UCU in different ways. In some embodiments, the sample in the UCU will be presented such that the emitted light interacts with the sample and is then reflected towards the light sensor. However, other ways of arranging the sample are likewise covered by the present invention. For example, the light emitter and the light sensor can be arranged on opposite faces of the UCU, and the UCU may comprise side faces transparent to the emitted light, such that the light travels linearly between the emitter and the sensor, passing through the sample contained in the UCU.

To measure urine samples, a variety of wavelengths, in that sense, both visible and infrared ranges, including near-, mid-, and far-infrared, allow measuring different types of components in the urine. Therefore, the wavelength of the light emitted by the light emitter can range between 360 nm and 2500 nm, and similarly, the wavelength measured by the light sensor can range between 360 nm and 2500 nm.

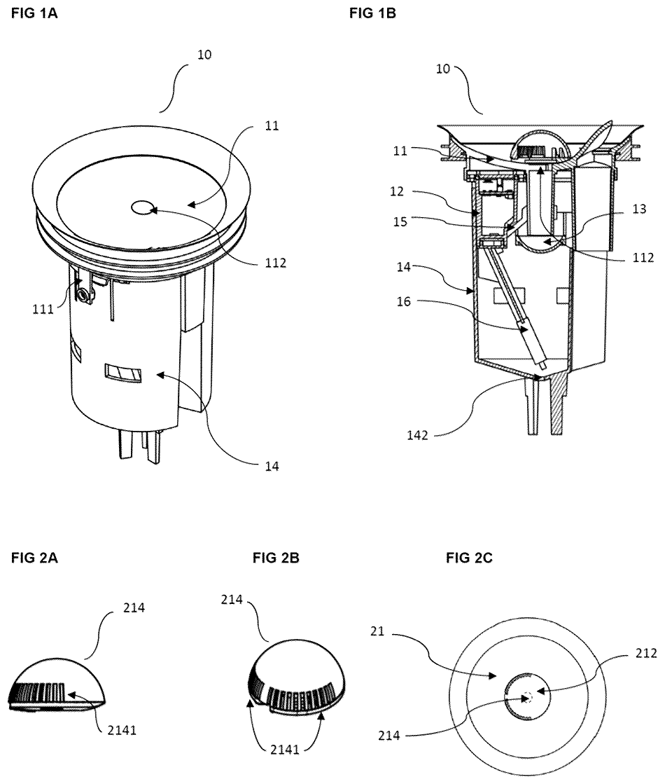

FIG. 1 shows a urinary measuring device (10) in a perspective view (FIG. 1A) and in a profile view with a cross-section (FIG. 1B). It can be seen that the device (10) comprises a funnel (11) with a main opening (112) which allows directing urine samples and water or sensor calibration liquids of the system deposited in the funnel (11) to the main opening (112). The device also has a casing for electronics (12) which contains the electronics and protects same from fluid damage and houses the electronics for an optical system (15) for the spectrophotometric measurement of the samples. In this sense, the device also has a urine collection unit (UCU) (13) which allows the samples to accumulate and be arranged such that spectrophotometric measurements can be performed on them by the optical system (15). Lastly, the device has a container (14) which stores excess sample from the UCU (13) and in turn has an outlet opening in its lower part (142) and an electrode system (16). However, in some embodiments, the device does not have the container (14) and/or the electrode system (16).

The perspective view of FIG. 1A only shows the funnel (11), the container (14), with the rest of the elements being concealed. Furthermore, it can be seen that the funnel (11) is arranged on the container (14). This embodiment has a fixing element (111) which allows the fixing thereof to other elements, such as the container (14). This fixing element may comprise one or more screws, nuts, openings, flaps, clips, grooves, friction systems, or any other element which allows fixing the funnel to another element of the optical measuring system. However, in other embodiments, the funnel (11) and the container (14) may be in contact. In other embodiments, the fixing element (111) may not be present. Furthermore, the funnel (11) has a circular shape, although in other embodiments the funnel (11) may have other shapes.

In the view with the cross-section of FIG. 1B, it can be observed that the funnel (11) provides the sample it collects to the UCU (13) through the main opening (112). Furthermore, it can be seen how the UCU (13) contains the urine sample such that it is exposed to the optical system (15). It can also be observed that, in this particular embodiment, both the casing for electronics (12) and the UCU (13) are contained inside the container (14) and between the funnel (11) and the container (14). However, in other embodiments, the casing for electronics (12) and the UCU (13) may not be contained inside the container (14). For example, the casing for electronics (12) may be located outside the container (14). Lastly, it can be seen that the electrodes (16) are arranged in the container such that they come into contact with the sample contained therein. In other embodiments, the electrodes can be arranged in an alternating manner and at different heights and orientations with respect to one another. Furthermore, the funnel (11) has a channel which guides the sample to the UCU (13) such that splashes are minimized, although in other embodiments the funnel (11) may not have this channel, or the channel may have other shapes.

In some embodiments, the main opening of the funnel has dimensions suitable for depositing urine in the UCU in a controlled manner, such that the formation of foam therein is reduced.

In a preferred embodiment, the funnel comprises a filter that at least partially covers the main opening configured to prevent the user form directly urinating into the UCU through the main opening. The filter is therefore designed to block the direct access of urine, with a reasonable margin from the directions in which urination may take place, so as to cover the different positions that the user may accidentally acquire. Generally, these positions are vertical and front positions, although depending on the urinary receptacle, other positions may be possible, and are therefore object of the present invention. This prevents the entry and the generation of foam. Additionally or alternatively, the filter comprises openings that are small enough so as to prevent the entry of unwanted objects to the main opening, and therefore to the UCU, which may interfere with the sample and the spectrophotometric analysis. Lastly, the filter can be designed with a shape which minimizes the risk of splashing on the user, for example with rounded shapes.

It should be highlighted that, by preventing direct access through the main opening, the speed at which the urine reaches the UCU is significantly reduced, which ensures a reduction of splashes that may reach the optical system. Furthermore, by covering the main opening, the amount of light accessing the UCU and the optical system is reduced, which improves measurements by the optical system.

FIG. 2 shows the profile view of a filter (214) (FIG. 2A), a perspective view of the filter (FIG. 2B), and a plan view showing the arrangement thereof in a funnel (21) (FIG. 2C). It can be seen that the filter (214) has a hemispherical shape which helps to reduce splashes. However, in other embodiments, the filter (214) may have other shapes. For example, in another preferred form, the filter may have an essentially flat shape. It can also be seen that the filter has a series of openings (2141) which prevent the passage of large objects and urine bubbles and other liquids to the inside. The passage of any object with a width greater than the width of these grooves would be stopped. Since the filter is arranged over the main opening (212) of the funnel (21), the filter effectively filters the passage of unwanted elements to the main opening. However, in other embodiments, the filter (214) may not have these openings (2141), leaving a single space for the passage of the sample to the main opening (212) or the openings (2141) thereof may have other shapes that are even different from one another, where the entry of unwanted substances can thus be prevented.

FIG. 2C shows the arrangement of the filter (214) in the funnel (21) in the central area thereof and covering a main opening (212). The filter (214) thereby prevents direct urination on the opening (212) and the entry of unwanted objects to same. However, in other embodiments, the main opening (212) may not be located in the central area of the funnel (21), in which case the filter (214) would not be located in said area either, but rather in the place where the main opening (212) were located. Furthermore, the filter (214) and/or the funnel (21) can in turn have different fixing mechanisms which allow the filter (214) to be fixed over the main opening (212) such that it does not move. The fixing mechanism may comprise one or more screws, nuts, openings, flaps, clips, grooves, friction systems, or any other element which allows fixing the filter (214) to the funnel (21) over the main opening (212).

Additionally, in a preferred embodiment, the filter may comprise a siphon mechanism configured to prevent gases from returning from the inner part of the funnel to the outside. The siphon mechanism may comprise different siphon systems. For example, the siphon may comprise interlinked elbows which generate a volume in a segment of the siphon that remains covered in water such that gases cannot run through the siphon. Alternatively, the siphon may comprise a membrane covering the passage of the siphon which yields to the pressure of the fluids running through the opening, but which is configured so that when there is no fluid running through the siphon, the membrane closes the siphon, preventing fluid communication of the inside of the funnel with the outside. In a preferred embodiment of the first aspect of the invention, the funnel further comprises a gasket in a part of the urinary measuring device which contacts the urinary receptacle for fluidically sealing a space between the urinary measuring device and the urinary receptacle. The gasket is therefore arranged at the height where the urinary measuring device is received by the inner walls of the urinary receptacle. In this sense, the location of the gasket is not limited by a specific height. However, for the purpose of preventing fluid accumulation, the gasket will preferably be arranged in the section of the funnel, more preferably in the upper part of the funnel. The gasket therefore provides the fluid it receives to the funnel, and the funnel in turn directs same towards the main opening thereof. Due to the sealing between the urinary receptacle and the gasket of the urinary measuring device, this gasket is fitted in the receptacle, preventing urine from running through anywhere other than through the urinary measuring device.

It should be highlighted that the gasket may have different shapes, including a circular shape, oval shape, or any other shape, including an irregular shape, in order to be adapted to the inner face of the urinary receptacle. Furthermore, the gasket can in turn be made up of different flanges, profiles, or protuberances which improve the fixing of the device to the urinal. Likewise, the gasket can in turn be made up of several gaskets arranged at different heights, providing an improved sealing level.

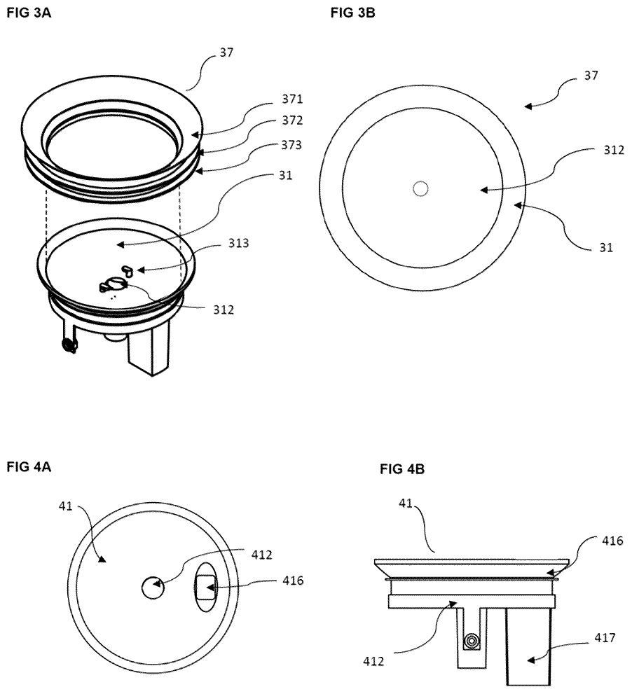

FIG. 3 shows a gasket (37) in the arrangement thereof with respect to a funnel (31), both in an exploded view (FIG. 3A) and in a plan view (FIG. 3B). It can be seen that the gasket (37) has a circular design and is configured to be arranged on the outside of the funnel (31). However, in other embodiments, the gasket may have any other shape that allows the adaptation thereof to the funnel (31) and to the inner face of the urinary receptacle. Furthermore, the shape of the inner part of the gasket (37) which interacts with the funnel (31) can, in some embodiments, be different from the shape of the outer part of the gasket (37) which interacts with the urinary receptacle, where the shape of both does not have to be the same. The gasket therefore ensures leak-tightness of the urinary measuring device at the level of the funnel. FIG. 3A shows fixing mechanisms (313) which allow a filter (not visible) to be fixed over the main opening (312) such that it does not move.

Furthermore, the gasket (37) comprises three sealing rings (371, 372, 373) at different levels, which ensure an improved fit with respect to the inside of the urinal and prevent urine from spilling out of the funnel (31). However, in other embodiments, the gasket (37) may comprise one or more gaskets a different levels or other mechanisms which ensures an improved fixing of the urinary measuring device to the urinary receptacle. Moreover, the gasket (37) is sealed to the funnel (31) by means of friction since the opening of the gasket (37) is smaller than the outer diameter of the funnel (31). Presumably, the gasket is preferably made up of a flexible material such that the sealing rings (371, 372, 373) push against the walls of the urinary receptacle when being inserted, so as to form a firm sealing. Furthermore, sealing is reinforced by the weight of the urinary measuring system, obtaining greater sealing when a sample is deposited on the urinary measuring device due to the weight of the sample. In other embodiments, the gasket and the funnel can interact by means of flanges, notches, or other fixing mechanisms other than the friction fixing mechanism. For example, in another preferred embodiment of the first aspect of the invention, the funnel (31) may comprise a screw mechanism which allows the coupling thereof to an external device comprising the gasket (37). In this sense, the funnel (31) can be coupled to a gasket (37) comprised as part of an adaptor element for a specific type of urinal, i.e., the gasket (37) may be part of a device with which the funnel (31) can interact through flanges, notches, or other fixing mechanisms other than the friction fixing mechanism.

In a preferred embodiment, the funnel further comprises an overflow opening configured to inhibit the sample from overflowing from the funnel, and a conduit configured to receive fluid from the overflow opening, with the conduit bypassing the UCU. After the discharge of water or cleaning fluid form a source of the urinary receptacle, the water flow received by the funnel increases considerably in comparison to a urination flow. Therefore, the main opening cannot account for the flow received and the water or fluid from the discharge is at risk of accumulating significantly on the funnel, thereby increasing the liquid elimination time during discharge. This overflow opening may also be incorporated directly in the toilet in a suitable place (and in this case, it would not be necessary to incorporate an overflow opening in the funnel). Although a gasket which seals the urinary measuring device may exist, it should be observed that the presence of fluids may cause damage to the different electrical and/or electronic part possibly comprised in the device. Therefore, the presence of an overflow opening in the funnel allows mitigating these problems. Its size is not limited, although it is preferably large enough so as to, along with the main opening, deal with the water flow coming in from the tank.

It should be highlighted that the overflow opening is preferably arranged in a more elevated position than the main opening. By being at a greater height, it is achieved that most, if not all, of the sample is collected during urination through the main opening which suitably directs same to the UCU for analysis. Furthermore, when the discharge of water or liquid occurs, the level of the liquid rises (as a result of a larger inflow than outflow in the funnel), and only then the overflow opening is used to evacuate water and thereby accelerate the elimination of water from the urinary measuring system. However, in other embodiments, the overflow opening may not be located at a height greater than the height of the main opening, and other mechanisms may be used to receive water in the case of a risk of overflow.

Moreover, it should be highlighted that the overflow opening provides the excess fluid through a conduit that serves to prevent exclusive collection by the UCU. Part of the deposited sample being redirected by the overflow opening has the advantage of accelerating the water elimination time of the urinary measuring system, in addition to reducing the exposure and use of the optical measuring system given the need to process less volume during discharges, thereby prolonging the service life thereof. However, there will always be a part of water coming from the part that goes through the main opening and is provided to the UCU, allowing the UCU to be cleaned, the systems to be calibrated, and the condition of the device to be understood (whether it contains urine or water, among other derived calculations).

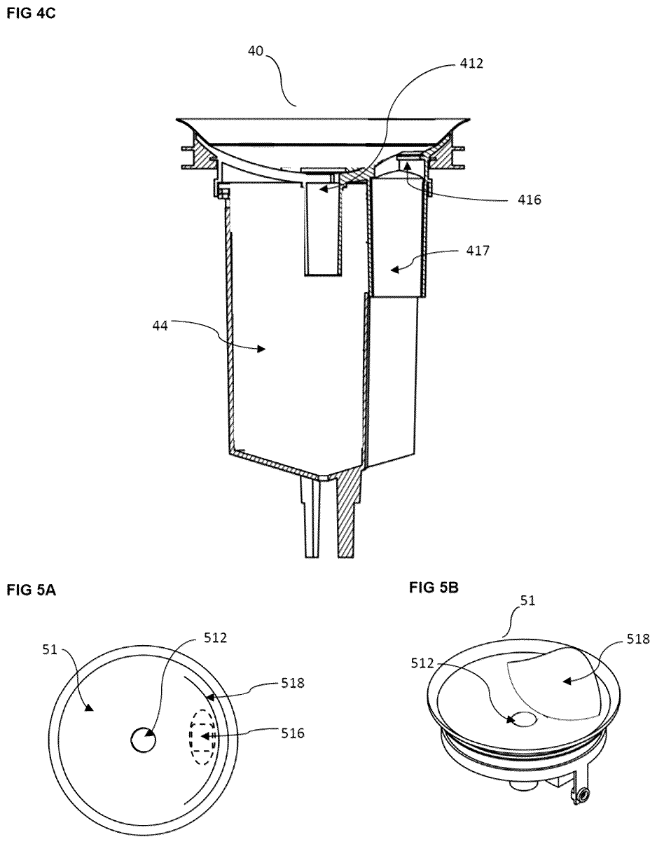

FIG. 4 shows a funnel (41) with an overflow opening (416) in a plan view (FIG. 4A), a profile view (FIG. 4B), and a profile view with a cross-section (FIG. 4C). It can be seen in FIG. 4A how the overflow opening (416) is arranged on one side of the funnel (41). The overflow opening (416) allows, in the case of overfilling the funnel (41), the possibility of passively relieving the amount of water stored therein. Since it is an overflow opening, when the level of the sample reaches the height of said opening, it is discharged through the overflow opening (416). However, in other embodiments, the overflow opening (416) can be placed in other locations which are not on a side.

Furthermore, FIG. 4B shows that the overflow opening (416) is arranged at a height greater than the height of the main opening (412). This allows the sample to be directed through the main opening (412) in urination situations, whereas the overflow opening (416) helps the evacuation thereof in water discharge situations. In other embodiments, the main opening (412) and the overflow opening (416) can be placed at any point of the funnel (41), and therefore the overflow opening (416) may not be located at a height greater than the height of the main opening (412).

Lastly, it can be seen in FIG. 4C how in the urinary measuring device (40), unlike the main opening (412), the overflow opening (416) provides fluids through a conduit (417) such that they are not measured. In some embodiments, the conduit (417) may not be present and/or may have other shapes and/or may redirect the fluids such that they were indeed measured by any other measuring system.

Optionally, in a preferred embodiment, the funnel further comprises a lid on the overflow opening configured to prevent the sample from flowing directly through the overflow opening. Despite the overflow opening preferably being arranged in a position which reduces the possibility of the user urinating directly, there is the possibility of the user urinating on the overflow opening, which would entail a partial or complete loss of the sample. By means of this lid, direct access to the overflow opening is not possible directly with a reasonable margin from the directions in which urination may take place, so as to cover the different positions that the user may accidentally acquire. Generally, these positions are vertical and front positions, although depending on the urinary receptacle and on the position of the overflow opening, other positions may be possible, and are therefore object of the present invention. However, the lid allows indirect access of the sample to the overflow opening, in the case where overflow is a possibility. In some embodiments, this means that horizontal access to the overflow opening is allowed, although this restriction may take different considerations according to the embodiment. Therefore, the lid does not prevent the evacuation of water when the fluid level in the funnel reaches the level of the overflow opening.

It should be highlighted that the shape of the lid is not limited in terms of size or form, provided that it prevents vertical and/or direct access of the fluid to the overflow opening, and allows horizontal access, i.e., access as a result of an overflow. Therefore, the lid can be curved to prevent splashes and/or with an angle that directs the fluid to the funnel or main opening or prevents it from being directed out of the funnel.

FIG. 5 shows a funnel (51) in a plan view (FIG. 5A) and in an isometric view (FIG. 5B), comprising a main opening (512) and a lid (518) covering the overflow opening (516). In FIG. 5B, it can be seen how the lid (518) is arranged such that it prevents direct urination on the overflow opening (516) (not visible), but still allows horizontal access thereto, allowing the access of the sample in the case of an overflow. Furthermore, the lid (518) tapers towards the main opening and has rounded shapes to reduce splashes. In this way, the lid (518) adopts the shape of a flap on the overflow opening (516). However, in other embodiments, the lid (518) can adopt other shapes which are not rounded or which preferably do not direct the fluids to the main opening (512). Furthermore, in other embodiments, the lid (518) and the filter of the main opening (512) can be attached or can work together in order to preferably provide the fluids to the main opening. Furthermore, in other embodiments, the lid (518) can be a separate element of the funnel (51) or can be provided from another element of the urinary measuring device, such as a gasket.

In a preferred embodiment of the first aspect of the invention, the light emitter and the light sensor are located in the casing for electronics for emitting and receiving light with respect to the UCU such that the planes perpendicular to the emission and measurement directions form an angle comprised between 120° and 180°. If the emitted light and the measured light have several directions, the central directions are taken. The optical measurement which allows taking several measurements of the sample involves interaction of the light emitted by the light emitter, the urine sample placed in the UCU, and the light sensor. The light emitted by the light emitter interacts with the sample, and the light sensor measures the light resulting from that interaction. In the cases where spectrophotometry is performed on the sample by means of incident light refraction and reflection, the position of these three elements is purposely pre-arranged at preferred angle and distances following Snell's law of electromagnetism. It follows, therefore, that the sample must therefore at least partially cover the UCU for spectrophotometry to be carried out.

Snell's law provides that the multiplication of the refractive index by the sine of the angle of incidence with respect to the normal is constant for any ray of incident light on the interphase between two different transmission media. Therefore:

n₁ sin(θ₁)=n₂ sin(θ₂)

for two media 1 and 2 with refractive indices n₁ and n₂, respectively. It is therefore possible to calculate the path of an incident light beam on a medium, as well as the exit thereof after being reflected at the bottom. Therefore, it is possible to calculate the angle at which the light emitter and the light sensor are arranged, the light sensor being capable of measuring the light reflected by the UCU containing the sample. Since the light emitter and/or the light sensor can be configured to emit/receive light over a range of angles, and the surface of the UCU can adopt a curved or concave shape, it can be seen that the light emitter and the light sensor can be oriented in a range of configurations and still be capable of detecting light which has passed through the sample.

Furthermore, it should be highlighted that the optical system is preferably arranged a distance from the UCU, such that the amount of splashes on the optical system is reduced, allowing a more durable system than if it were submerged or in direct contact, since deterioration of the optics due to lime and dirt is prevented.

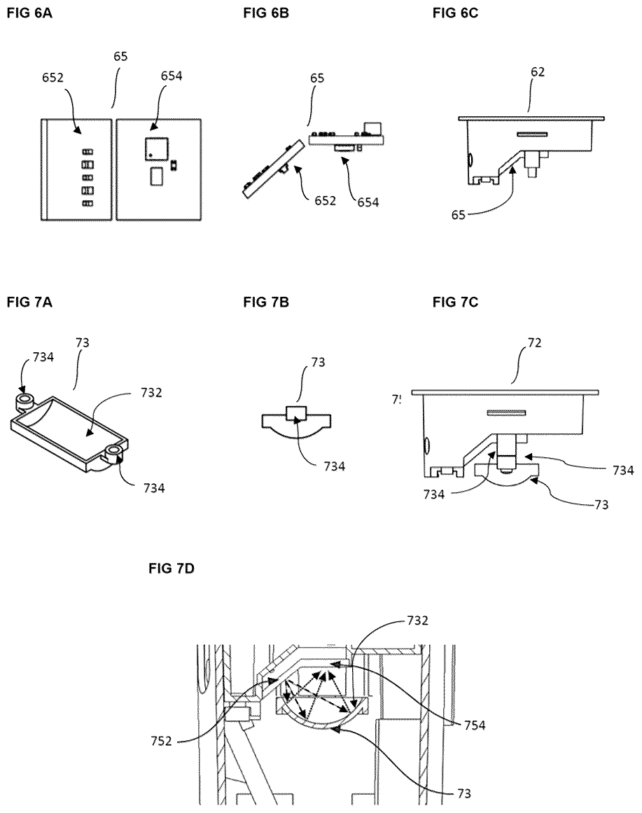

FIG. 6 shows an optical system (65) in a plan view (FIG. 6A) and a profile view (FIG. 6B) and a casing for electronics (62) housing the optical system (65). It can be seen in FIG. 6A that the optical system comprises a light emitter (652) which in turn comprises several LED diodes which allow emitting light to perform a spectrophotometry test. In other embodiments, the light emitter (652) may comprise other light emitters in different combinations. Similarly, it can be seen that the optical system (65) comprises a light sensor (654) which in turn comprises several sensors which allow detecting light coming from a sample in a plurality of wavelengths over a spectrum to be analyzed, after having interacted with the sample to perform a spectrophotometry test. In other embodiments, the light sensor (654) may comprise one or more sensors of different types (different methods for measurement, sensitivity to different frequencies). It can be seen in FIGS. 6B and 6C that the light emitter (652) and the light sensor (654) are arranged at a predetermined angle. In other embodiments, the predetermined angle can be different. Furthermore, it can be seen particularly in FIG. 6C that the casing for electronics (62) containing the optical system (65) is configured to arrange the light emitter (652) and the light sensor (654) at the desired predetermined angle. Although not present in this embodiment, the optical system (65) of other embodiments may also comprise a protector which protects the exposed face of the optical system against the sample splashing from the UCU.

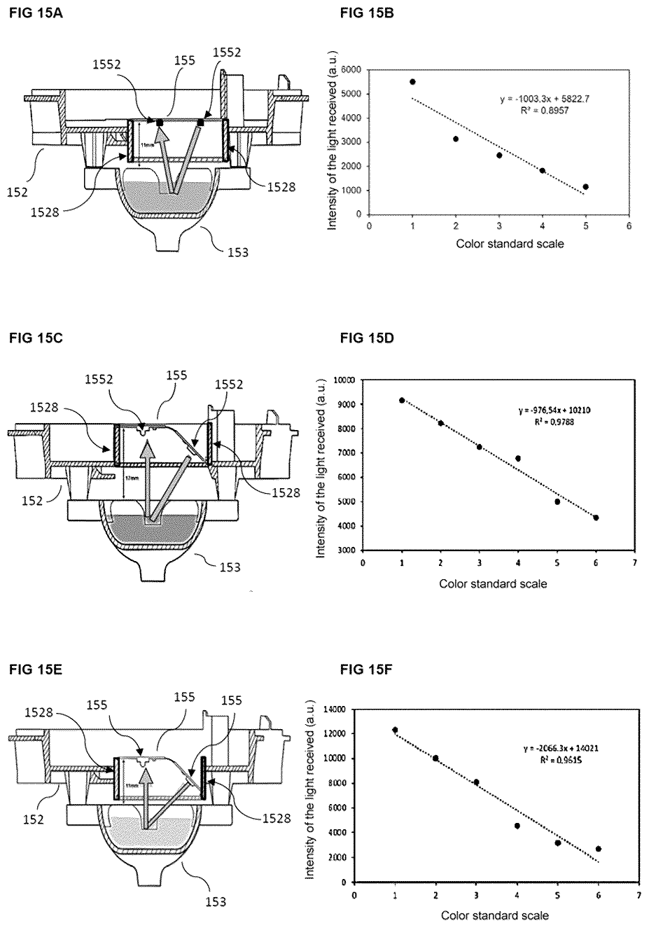

In another preferred alternative embodiment of the first aspect of the invention as shown in FIG. 15A, the light emitter (1552) and the light sensor (1554) of the optical sensor (155) are located in the casing for electronics in order to emit and receive light with respect to the UCU (153) such that the planes perpendicular to the emission and measurement directions form an angle of preferably 180°, that is, with the same direction and sense. If the emitted light and the measured light have several directions, the central directions are taken. The optical measurement which allows taking several measurements of the sample involves interaction of the light emitted by the light emitter (1552), the urine sample placed in the UCU (153), and the light sensor (1554). The light emitted by the light emitter (1552) interacts with the sample, and the light sensor (1554) measures the light resulting from that interaction. In the cases where spectrophotometry is performed on the sample by means of incident light refraction and reflection, the position of these three elements is purposely pre-arranged at preferred angle and distances following Snell's law of electromagnetism as described above.

FIG. 15 shows several alternatives of a casing for electronics (152) housing the optical system (155) and an UCU (153). It can be seen that the optical system comprises a light emitter (1552) which in turn comprises several LED diodes which allow emitting light to perform a spectrophotometry test. In other embodiments, the light sensor (1554) may comprise one or more sensors of different types (different methods for measurement, sensitivity to different frequencies). It can be seen in FIGS. 15C and 15E that the light emitter (1552) and the light sensor (1554) are arranged at a predetermined angle. In other embodiments, as shown in FIG. 15A, the predetermined angle can be different, for example, with light being provided to perform a spectrophotometry test forming an angle of 180°. In this sense, the present embodiment is not limited to a specific type of angle between the light emitter (1552) and the light sensor (1554).

Furthermore, it can be seen in FIGS. 15C and 15E that the optical system (155) is arranged at different distances from the UCU (153). In other embodiments, the optical system (155) can be arranged in direct contact with the UCU (153), at a distance far away from the UCU (153) as shown in FIG. 15C, or at a distance close to the UCU (153) as shown in FIG. 15E. Advantageously, this allows different emitter and receiver technologies (different methods for measurement, sensitivity to different frequencies) which require and/or allow the use of different distances between the optical system (155) and the UCU (153) to be used in the light device to perform a spectrophotometry test. In this sense, the present embodiment is not limited to a distance between the light emitter (1552) and the light sensor (1554).

Furthermore, it is observed that the optical system (155) is comprised between optional walls (1528) of the casing for electronics (152) configured to prevent light from scattering in unwanted directions. Advantageously, this allows directing and maximizing the projection and reception of light between the light emitter (1552) and the light sensor (1554). Preferably, the walls (1528) define a surface in the direction of measurement by the optical system (155) to and from the UCU (153).

FIGS. 15B, 15D, and 15F show the different standard curves of the optical systems (155) of FIGS. 15A, 15C, and 15E, respectively, in comparison with a color standard. The color standard is made up of a color scale where the value 0 represents transparency and the value 6 represents the most intense color which therefore absorbs a higher amount of light. Therefore, a larger number represents a more concentrated urine. As can be seen, in all the cases, regardless of the angle at which the light emitter (1552) and the light sensor (1554) are arranged and of the distance between the optical systems (155) and the UCU (153), the light sensor (1552) is capable of measuring the light received from the UCU (153) with great precision in all the cases, such that the standard curve has an R² fitting of at least 0.89.

In a preferred embodiment, the casing for electronics comprises a main body, a cover, and a gasket configured to fluidically seal the interface between the main body and the cover. The gasket allows fluidically sealing the main body and the cover, preventing the entry of fluids into same in the event that a fluid comes into contact with the casing for electronics. This therefore provides greater durability to the electronic system.

In a preferred embodiment, the casing for electronics comprises a transparent protector arranged for protecting the light emitter and/or the light sensor against the sample splashing from the UCU. Since the light emitter and the light sensor are directed towards the UCU, it is possible that certain splashes of the sample may reach these sensors from the UCU. Therefore, the transparent protector allows protecting the light emitter and the light sensor from splashes, prolonging their service life and preventing erroneous readings, as would occur if a drop of sample were to partially or completely block the light emitter or the light sensor.

The transparent protector can be formed from any material which allows the light to be transmitted to the UCU from the light emitter and allows the light reflected from the UCU to reach the light sensor, including any of the following: styrene methacrylate (SMMA), polycarbonate, poly(methyl methacrylate) (PMMA), quartz, amorphous quartz, glass, chemically reinforced glass (such as Gorilla Glass or Xensation Schott glass), or sapphire crystal. The exposed face of the transparent protector can be covered with a coating to prevent the formation of bacteria (for example, an anti-bacterial coating) and/or to prevent the degradation of the protector as a result of the splashes.

In a preferred embodiment, the UCU comprises a surface arranged for maximizing the reflection of an incident light and which in turn comprises a material selected from one of the materials from the following list: ceramic, crystal, glass, porcelain, aluminum, polymers such as POM or a derivative of these materials. The surface of the UCU can also be treated with different agents and/or processes such as, but not limited to, specific coatings or paints causing the surface to have optimal whiteness to perform the analysis. The selection of these materials allows an UCU that is durable in the face of various aggressive agents (lime, urea, detergents, etc.) and facilitates the directing of the light coming from the light emitter to the light sensor. Furthermore, the shape of the UCU may favor the reflective effect of these materials, such that incident light is directed more directly to the light sensor.

FIG. 7 shows a urine collection unit (UCU) (73) in a perspective view (FIG. 7A) and in a profile view (FIG. 7B), a casing for electronics (72) housing an optical system (75) and with the UCU (73) being arranged in the central area thereof, where a funnel (not visible) would deposit the sample (FIG. 7C), and a profile view (FIG. 7D) of the interaction of the light on the surface of the UCU (732). It can be seen in FIGS. 7A and 7B that the UCU (73) has a concave inner surface (732), which allows increasing the amount of light reaching the light sensor, increasing the sensitivity of the system. Other embodiments may not have this surface or may have a surface with another shape different from that of this embodiment. The UCU (73) comprises one or more fixing elements (734) which allow fixing the UCU (73) to other components of the apparatus. In the illustrated embodiment, the fixing elements (734) allow the fixing thereof to the casing for electronics, whereas in other embodiments, the fixing elements (734) may allow the fixing thereof to other elements of the apparatus, such as the funnel. These fixing elements may comprise one or more screws, nuts, openings, flaps, clips, grooves, friction systems, or any other element which allows fixing the UCU to another element of the urinary measuring device.

Furthermore, it is seen in FIG. 7C that the UCU is positioned a certain distance from the optical system (75) contained in the casing for electronics (72), which prevents splashes and deterioration as a result of lime and the formation of films on the lenses. In other embodiments, the UCU may be positioned in contact with the optical measuring system. It can also be noted how the optical system (75) establishes a different angle with the UCU (73) for the light emitter and for the light sensor such that the light emitted by the light emitter is reflected on the sample contained in the UCU and received by the light sensor. It can also be seen how the fixing element (734) allows the UCU to be fixed to the casing for electronics in this particular figure. Although not present in this example, in other embodiments, the casing for electronics (72) may comprise a transparent protector protecting it against the sample splashing from the UCU.

It is shown in FIG. 7D how the UCU (73) is positioned and configured to converge all the light beams coming from the light emitter (752) in the light of the light sensor (754). In this preferred embodiment, the surface of the UCU is in the form of a reflective lens and the light sensor is positioned close to or at the focus point, in order to maximize sensitivity. This improves light detection by the light sensor. In other embodiments in which light reflection is used to receive light in the light sensor, the position and shape of the UCU may or may not achieve the convergence of light beams in the light sensor through other means, as long as part of the reflected light reaches the light sensor. The importance of the UCU having a surface (732) that reflects light sufficiently is also observed.

In a preferred embodiment, the UCU comprises a series of side recesses (not shown) which allow establishing a maximum urine level in the UCU that is lower than the maximum height thereof, such that the UCU is not filled up completely, but rather there is always an upper section of the UCU that has no urine. Advantageously, this allows the urine level to always be the same level, facilitating homogenous measurements by the optical system, while at the same time eliminating urine from the UCU in a controlled manner.

In an even more preferred embodiment, the UCU comprises an outer surface in the lower part of said side recesses which allow controlling the running of the urine and fluids on the UCU during the outflow thereof. The UCU preferably comprises an outer surface the base of which comprises a rounded shape with a truncated cylinder at the end thereof, such that the urine and fluids exiting the UCU through the recesses (not shown) run along the outside of the UCU towards said truncated cylinder, where they are finally eliminated from the UCU to subsequent stages of the urinal. Advantageously, this allows the flow from the UCU to the subsequent stages of the urinal to be carried out in a controlled manner.

In a preferred embodiment, the urinary measuring device further comprises:
d. A container for collecting the fluid overflowing from the UCU, comprising an outlet opening; and
e. A plurality of electrodes extending inside the container by means of arms.

The device may comprise a voltage source connected between the electrodes configured to apply a voltage signal between the electrodes and a current meter configured to measure the current between the electrodes resulting from a voltage signal applied by the voltage source. The device may comprise an impedance meter connected to the electrodes, the impedance meter configured to measure the impedance of the fluid present in the container between the electrodes.

In this preferred embodiment, the container is arranged such that the fluid overflowing from the UCU is collected by the container. Therefore, the container is capable of housing a fluid. Moreover, the outlet opening is arranged so as to allow the exit of the collected fluid, preferably in the lower part of the funnel, such that the sample contained in the container can be evacuated entirely through the outlet opening. The electrodes can be arranged in any part of the container where the sample will be collected, preferably in a position close to the position of the outlet opening. In some embodiments, the electrodes can be arranged by means of a plurality of arms extending from another element of the device, such as the casing for electronics. In a preferred embodiment, the outlet opening of the container is configured to allow a predetermined outflow that is smaller than that of the main opening of the funnel. By means of the design of the outlet opening of the container, the flow of the fluid running through the container can be controlled. By having a flow that is smaller than that of the main opening of the funnel, there is also an opening with a flow smaller than that of the inflow of the funnel from the UCU as a result of an overflow. Therefore, when receiving a fluid from the UCU, an effect of filling the container with the incoming fluid, either the urine sample or water from the water tank, occurs.

The advantage of knowing the size of an outlet opening is that it can be used to calculate the total volume of fluid running through the opening. If the speed of the fluid through said opening is known and the time (t) it takes for all the fluid to run is calculated, the total volume of fluid which has run through the opening can be obtained. In this sense, it is possible to predict the speed of a fluid (v) running through an opening for a certain fluid column having height (h) by means of Torricelli's law defined as:

ν=\sqrt{2gh}

where g is the universal gravitational constant. One of the conclusions of this equation is that the flow changes with the height of the fluid column. Therefore, it is necessary to take additional considerations for obtaining measurements which take into account this change in height.

If certain assumptions are made, such as the opening being arranged in the lower part of a cylinder with a base having an area A, where the opening covers an area a, and a height h, the outflow speed v can be determined as:

$$v = \frac{dx}{dt} = \sqrt{2 gh}$$

where dx is the equivalent height of a fluid cylinder having an area a formed by the fluid contained in the container having an area A. Therefore, the following can be defined:

$$dx = \frac{A}{a} dh ,$$

from which it follows that

$$\frac{dx}{dt} = \frac{A dh}{a dt} = \sqrt{2 gh}$$

By solving and integrating, the time elapsed between an initial height hi and a final height hf is obtained as follows:

$$t = \frac{2 A}{a \sqrt{2 g}} ( \sqrt{{h}_{i}} - \sqrt{{h}_{f}} )$$

and given that the change in height of the water column is related to the volume V as follows:

$$Δ h = \frac{Δ V}{h}$$

it follows that:

$$Δ t = \frac{2 A}{a \sqrt{2 g}} \sqrt{\frac{Δ V}{h}}$$

Therefore, it is possible to establish a relationship between the volume of the liquid stored in the cylindrical container having an area A and the time elapsed in emptying a fluid column having a height h through an opening having an area a.

It is possible to establish this relationship for other shapes of the container, but additional steps may be required. For example, although linking the volume with height is more complex for a bowl-shaped container in which the outlet opening is in the center, in practice this shape provides a volume-time relationship close to linearity. This is due to the fact that, although the pressure exerted by the fluid column is lower when emptying the container, the bowl shape maintains a constant pressure in the outlet opening. It is even possible to define container shapes which ensure a constant outflow, like in the case of outflow water clock.

Therefore, it follows that, given a container with a defined shape, it is possible to determine the volume flowing through an opening made in the container, given a known water height and the time taken for emptying same. In the case of a design which ensures a constant flow, the height would be negligible. It should also be highlighted that, for a container with an arbitrary shape, the relationship between the time it takes to empty the container and the total volume can be determined empirically through observations which link the time elapsed with the volume of liquid which has exited the container and stored in a lookup table. The system can then compare the time elapsed in the lookup table and determine the total volume. Alternatively, the total volume can be determined through predetermined equations and algorithms taking into account a known volume, the flow rate, the outlet opening, and the emptying time. In this manner, the urinary measuring system can then correlate the time elapsed through the algorithms saved in a memory (for example, in a memory of the device or in an external memory such in the cloud) in order to determine the total volume. Alternatively, a machine learning algorithm which is trained can be used to link total urination time with total urination volume for a container with a specific shape (the machine learning algorithm can be trained using a training data set comprising the time it takes to empty the specific container for a plurality of known fluid volumes). Once the total volume and the total urination time are calculated, the urination flow rate can also be calculated as wall. In the case of hydration, the present system determines the specific level of hydration through electrical impedance as indicated in relation to FIG. 14.

In a preferred embodiment in which the container has no walls with regular shapes or in which the shape of the container hinders the measurement of the total volume, the container comprises in its base longitudinal walls around the outlet opening, configured to generate a regular volume in the form of the container around the outlet opening and in which the plurality of electrodes can be placed. Advantageously, this allows the fluid to flow through the outlet opening from said volume which, due to the longitudinal walls, allows a more precise and regular measurement of the total volume by means of the plurality of electrodes. More preferably, the longitudinal walls define a cylinder.

In the embodiments in which the UCU comprises a series of side recesses and an outer surface in the lower part of said side recesses, which allow controlling the running of the urine and fluids on the UCU during the outflow thereof, the outer surface of the UCU directs the urine flow from the UCU to the container defined by the longitudinal walls of the container. Even more preferably, the UCU directs the urine flow from the UCU to the container through a rounded shape with a truncated cylinder at the end thereof, such that the urine and fluids exiting the UCU through the recesses (not shown) run along the outside of the UCU to said truncated cylinder, where they are finally directed to the container.

The filling of the container allows the electrodes arranged therein to come into contact with the fluid. This in turn allows taking, by means of the electrodes, additional measurements of the fluid, either urine sample or water, related to its electrochemistry. In some embodiments, the urinary measuring device further comprises an impedance meter connected between the electrodes which allows measuring the impedance of the medium between the electrodes. The impedance meter can be any type of impedance meter for measuring the impedance between the electrodes. The impedance meter can transmit a voltage signal (which can be an AC or DC signal, preferably an AC signal) through one of the electrodes and measures the impedance profile of the medium through the reception of the same signal by the other electrode. The signals may have a frequency preferably between 1 and 10M Hz or 1 and 100 MHz, preferably sinusoidal frequencies.

In some embodiments, the impedance meter measures the complex impedance between the electrodes. Complex impedance can be measured by calculating the relationship between the complex representation of the sinusoidal voltage signal through the electrodes, and the complex representation of the current flowing through the electrodes. Any type of complex impedance meter can be used.

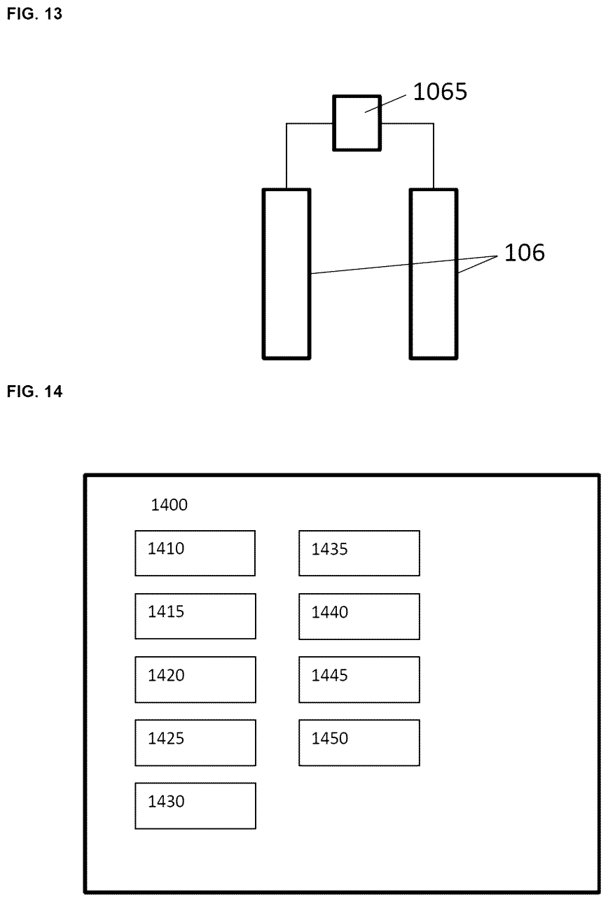

FIG. 13 shows the arrangement of an impedance meter according to one or more embodiments. The impedance meter 1065 is arranged between the electrodes to measure the impedance of the medium arranged between the electrodes 106. In other embodiments, several electrodes can be connected to several impedance meters and/or several impedances can share one and the same electrode by way of reference.

The electrodes are preferably monitoring the impedance of the medium either intermittently or continuously. When the impedance of the medium in which the electrodes are located is greater than a threshold, i.e., when the impedance thereof drops below a certain value, a sample can be defined as having come into contact with the electrodes, and therefore as being present. Likewise, it is determined that a sample is not present when the impedance between the electrodes is above a certain threshold. This allows making decisions depending on whether or not a sample is present.

Furthermore, a series of electrodes can be arranged at different heights, where the device comprises one or more impedance meters configured to measure the impedance between the electrodes at a specific height. This allows precisely detecting the sample level in the container, as different electrodes report different impedances. Furthermore, it allows knowing the estimated container emptying time before the container finishes emptying, or if the container is filled in midway through the process, detecting it. The various electrodes can be arranged in the same arm or in different arms. One electrode can serve as a reference electrode common to several measurement electrodes.

In a preferred embodiment, the container has a cylindrical shape and is configured to provide disponer a fluid through its outlet opening at a constant speed.

In a preferred embodiment, the container further comprises a side outlet opening, arranged at a greater height than that of the outlet opening and than that of the plurality of electrodes. Given that, in some embodiments, the outlet opening of the container may allow a flow lower than the inflow, an evacuation system which prevents excessive filling of the funnel can be provided, affecting the optical system if it is arranged inside the container. This effect is achieved by means of one or more outlet openings on the side of the container. Furthermore, the height of these side openings is selected such that it is lower than the height of the optical system, but higher than the location of the electrodes. In this way, the side openings do not interfere with the measurement function of the electrodes and protect the electronics from excessive exposure to the fluid.

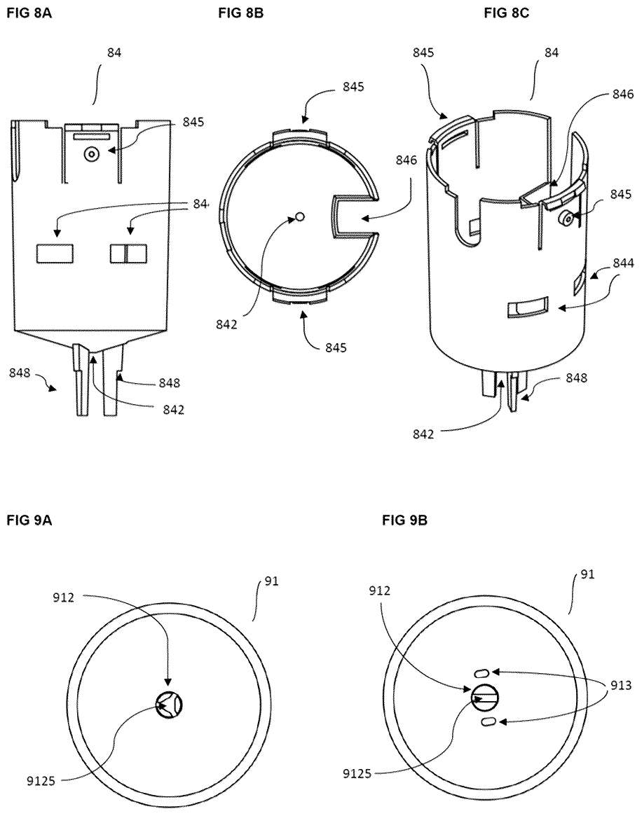

FIG. 8 shows a container (84) in an elevational view (FIG. 8A), in a plan view (FIG. 8B), and in a perspective view (FIG. 8C). It can be seen in FIG. 8A that the container (84) has two side outlet openings (844) arranged at a mid-height of the container. These two openings allow the sample contained therein to be evacuated in the case where the level is high, such that the sample contained therein is prevented from reaching the electronic and/or measuring systems. The height of these openings is limited by the casing for electronics, the optical system, and the UCU, since the container (84) is designed for housing all these elements; and it is always located at a height greater than the height of the outlet opening (842). However, in other embodiments, the container (84) may not have these side openings (844) or may have only one or more than two side openings (844). Furthermore, the height and design of the openings may vary according to the embodiment, which may not house any of the elements such as the casing for electronics, the optical system, and/or the UCU.

It should also be noted that the base of the container (84) has a conical shape in the form of a funnel which allows it to direct fluids to the outlet opening (842). Other embodiments may not have of any shape in the base of the container (84) which will help them to direct fluids. Likewise, the outlet opening (842) may not be arranged in the center of the base of the container (84), or even in the lowest part thereof. In the container (84), a series of flanges (848), each of them comprising a support surface (8482), help to securely fix the container (84) to a urinary receptacle by means of the interaction of the support surfaces (8482) with a lower surface of the urinal. In other embodiments, the container (84) may have another type of systems (848) which help to fix or support same on the urinary receptacle, or it may dispense with said systems. If the urinary receptacle is a toilet, the anchoring systems may comprise flanges configured to secure the device to the edge of the toilet or any other system which allows fixing the device in the toilet.

FIG. 8B shows the plan view of the container (84) having the outlet opening (842) in the center thereof. However, in other embodiments, the outlet opening (842) may not be arranged in the center of the base of the container (84). Furthermore, the presence of a recess (846) extending along the entire height of the container (84), which is configured to accommodate the conduit (417) conducting the fluids coming from the side opening of a funnel bypassing the UCU and the container, should be highlighted.

It can be seen in FIG. 8C that the container (84) also has a series of fixing elements (845) which allows the fixing thereof to other elements such as, for example, a funnel. These fixing elements may comprise one or more screws, nuts, openings, flaps, clips, grooves, friction systems, or any other element which allows fixing the container (84) to another element of the urinary measuring device. The fixing elements (845) can be compressed to release the flanges of the casing for electronics. The recess (846) which allows accommodating the conduit that allows the sample to bypass the UCU and the container (84) can also be seen.

In a preferred embodiment, electric cables extend from the arms to the casing for electronics through a gasket, in order to form an electrical connection between the electrodes and the electronics in the casing for electronics.

In a preferred embodiment, the funnel comprises a handle configured to facilitate the extraction of the urinary measuring device by means of an extraction device. The handle is arranged on the upper surface of the funnel so that it is accessible when the device is installed in the urinal or toilet. For the extraction of the urinary measuring device, various extraction devices with hooks can be used, such that the operator removing the urinary measuring device does not come into direct contact with the device, minimizing the risk of exposure to pathogens. To that end, a handle arranged in the funnel allows securely grabbing the urinary measuring device with the extraction device, overcoming the weight thereof and the resistance caused by the friction of the gasket with the inner surface of the urinary receptacle. Preferably, the handle is arranged on the main opening of the funnel, but it can be located in any part of the funnel which is accessible from the upper surface of the funnel.

FIG. 9 shows two embodiments (FIG. 9A and FIG. 9B) of a funnel (91) in a plan view, in which the main opening (912) has a handle (9125) which allows extracting the urinary measuring device by means of an extraction device. It is observed that the handle (9125) is a representative mode and can adopt different shapes in other embodiments. Therefore, in FIG. 9A, the handle has a triangular shape, whereas in FIG. 9B, it has a rectangular shape. For example, the handle can adopt bulk shapes projecting from the plane of the surface of the funnel. Likewise, the handle (9125) can be located in different location in the funnel (91), where the location thereof is not limited to the main opening (912). Therefore, the handle (9125) can be comprised in any location in the funnel (91). In some embodiments, it is also possible for the handle (9125) to be made up completely of another element of the funnel (91), such as a covering or the main opening (912) itself. Lastly, FIG. 9B shows the funnel (91) having two fixing mechanisms (913) which allow fixing a filter (not visible) over the main opening (912) to prevent direct urination and the entry of objects and urine foam through the main opening.

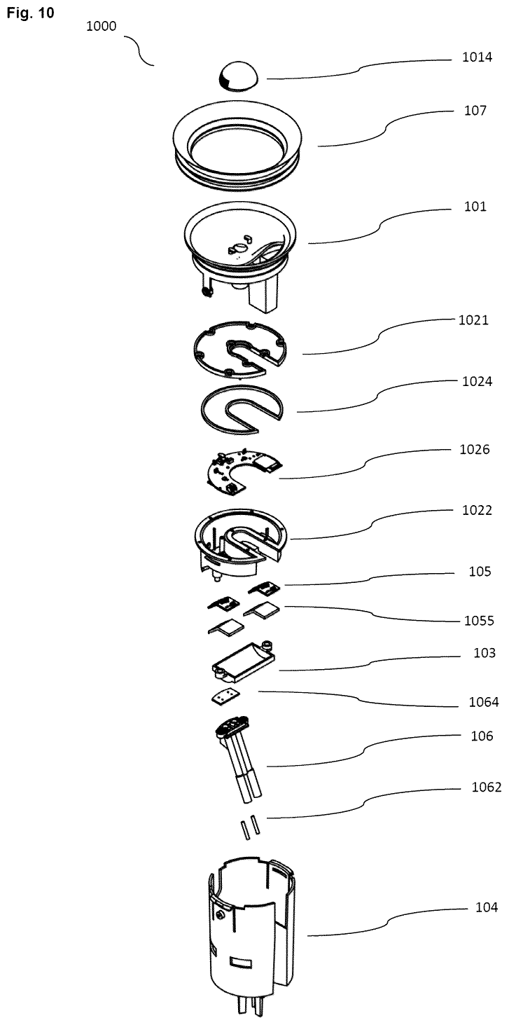

FIG. 10 shows an exploded view of a urinary measuring device (1000) according to an embodiment. The urinary measuring device (1000) has a filter (1014) over the main opening of the funnel (101) which prevent the entry of substances which may damage the device through the main opening. In some embodiments, this filter is not provided. The filter can be like the one described in reference to FIGS. 2A-2C.

The device also has a gasket (107) in the outer part of the funnel configured to prevent the spillage of samples between the funnel (101) and the urinary receptacle. This gasket can be like the one described in reference to FIGS. 3A and 3B. Some embodiments are not provided with the gasket (101) of the funnel (101).

Furthermore, it is observed that the device has a casing for electronics made up of an upper cover (1021), an isolating gasket (1024), and a base (1022). This casing for electronics can be like the one described in reference to FIG. 6C. In some embodiments, the casing for electronics is not provided with the isolating gasket and/or is formed by more or less pieces. The casing for electronics houses in the inside thereof a controller (1026), although not limited thereto, and in the lower part thereof an optical system (105). Some embodiments do not have a controller (1026). For example, the casing for electronics can be configured to transmit and receive control signals and/or to receive sensor data from the light sensor and/or the impedance meter in order to control the electronic components of the device through an external controller (for example, by means of a receiver or by cabled connections to the external controller). The optical measuring system has protectors (1055) which protect the optical measuring system from possible splashes. These protectors are optional in some embodiments.

The urinary measuring device (1000) also comprises a urine collection unit (UCU) (103) fixed to the casing for electronics through bolts. This urine collection unit can be like the one described in reference to FIGS. 7A-7D. The UCU (103) allows collecting samples coming from the main opening of the funnel (101). In some embodiments, the UCU (103) can be fixed to other elements of the device and by other means, such as those described above.

Moreover, the device comprises two electrodes (1062) contained in one arm (106) attaching same to the casing for electronics. The arm (106) allows the electrodes (1062) to be arranged in a preferred position, and the electrodes (1062) allow the signal circulating between both to be measured. Although this embodiment shows two arms (106), other embodiments may have only one arm, more arms, or may not have any arms at all. Furthermore, the electrodes (1062) of the arm (106) can be arranged at different heights and have different shapes according to the embodiment. In this embodiment, the arms (106) comprise a gasket in the attachment thereof with the casing for electronics which prevents the entry of fluids therein. In other embodiments, the gasket (1064) is not provided.

Lastly, the urinary measuring device (1000) has a container (104) collecting samples that overflow after the filling of the UCU (103) and arrange it such that the electrodes (1062) can be submerged when a sufficient amount of sample is introduced. In some embodiments, the container (104) is not provided. The container can be like the one described in reference to FIGS. 8A-8C.

In a preferred embodiment, the casing for electronics further comprises a controller configured to control the light emitter, the light sensor, and optionally the impedance meter, and wherein the controller is made up of a processor configured to process information from the light sensor and the impedance meter. The casing for electronics can contain an internal energy source such as a battery, or can be connected to an external energy source, and the controller selectively directs the energy to the different components. Alternatively, the casing for electronics comprises a communication interface for wired or wireless communication with an external processor and/or a controller which are configured to process and/or control information from the optical system and/or the electrodes.

The controller is configured to control the optical system by means of an interface (the light emitter and the light sensor) and the electrodes, and to receive information from the optical system and the electrodes and to process said information. Therefore, the processor is configured to control when to activate the light emitter, when the light sensor detects the light it receives, and when to activate the impedance meter (if measurement is taken intermittently). Similarly, the processor receives information about the light detected by the light sensor and about the impedance of the medium. Lastly, the processor is configured to process the information received in different ways: it calculates parameters of the information obtained and sends signals to the components by means of the interface, turning the components on and off.

Optionally, the controller may comprise a memory. The memory is configured to store the instructions executed by the processor, as well as to store information received by the processor and information processed by the processor. Furthermore, the processor can be configured to be connected to a computer external to the urinary measuring device, such as a cloud server, in order to transmit and receive information.

All these connections can be made by electric or electronic means through cables of any type and/or as telecommunication protocols such as WiFi, Bluetooth, NFC, among others.

It should be highlighted that the processor may comprise one or more processing units, such as a microprocessor, a GPU, a CPU, a multicore processor, or the like. Similarly, the memory may comprise one or more volatile or non-volatile memory devices, such as DRAM, SRAM, flash memory, read-only memory, ferroelectric RAM, hard disk drives, floppy disks, magnetic tapes, optical disks, or the like. The controller can be implemented in software (a computer program), hardware (a physical device), or any combination for the purpose of executing the operation sequences disclosed herein.

In a preferred embodiment of the first aspect of the invention, the urinary measuring device further comprises a valve of the urinary receptacle configured to control the outflow of water from a water source to the urinary receptacle, wherein the device is configured to control the valve. The valve can be arranged both inside a tank in an upper wall of a urinal and in the shutoff cock for shutting off the water feeding the tank, so as to have control over when the water from the tank is deposited in the urinary receptacle. In this embodiment, the processor of the controller is preferably configured to control the valve, so it can control when to open and close the valve. Similarly, if the controller comprises a memory, the memory is preferably configured to store the instructions of the processor which allow it to control the valve. It should be highlighted that the valve can be of different types, such as solenoid valves or pneumatic control valves. In the last case, the device also comprises a pneumatic pump system which allows controlling the pneumatic control valve. In some embodiments, the urinary measuring device is configured to open the valve when it detects that sample measurement has ended.

In another preferred embodiment, the urinary measuring device further comprises a user interface configured to receive identification or anonymous information of a user (i.e., information that does not identify the user) and to show information about the condition of the sample to the user. The user interface can therefore receive a user input which identifies the user and provide said user with information of interest throughout the urination process, analysis, or at the end of the process. In this embodiment, the processor of the controller is preferably configured to receive and transmit information of the user interface, so it can receive information about the identity of the subject and transmit information about the urine analysis performed, as well as the condition thereof. Similarly, if the controller comprises a memory, the memory is preferably configured to store the instructions of the processor which allow it to communicate with the control unit.

The user interface may comprise several means for interacting with the user such as a screen, a touch screen, a haptic interface, a microphone, a camera, a button, a keyboard, and an RFID sensor, among others. Furthermore, the user interface may also comprise mobile and/or remote means for interacting, such as one or more Bluetooth antennas, WiFi antennas, among others.

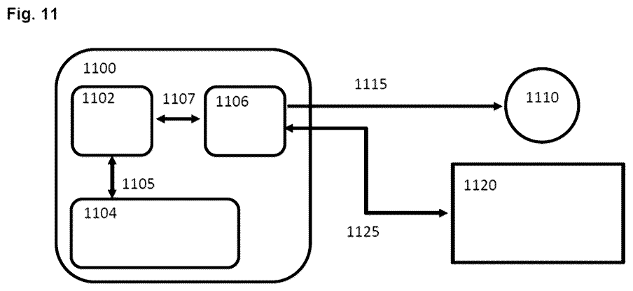

FIG. 11 shows a diagram of the interaction between a controller (1100), a solenoid valve (1110), and a user interface (1120). As can be seen, the controller (1100) comprises a processor (1102) and a memory (1104). In other embodiments, the controller (1100) may not comprise the memory (1104). It can also be seen that it is the processor which executes the orders (1115) controlling the solenoid valve, and which sends and receives information (1125) to and from the user interface. Moreover, the memory (1104) sends and stores information (1125) to and from the processor (1102). The processor also sends and receives information and control signals to and from the optical system and/or the electrodes (1107) through the control interface (1106). Likewise, in other embodiments, the processor can also send and receive information to and from an external computer, such as a web server. All these connections can be made by electric or electronic means through cables of any type and/or as telecommunication protocols such as WiFi, Bluetooth, NFC, among others. In other embodiments, it is possible for the controller (1100) to control more than one solenoid valve (1110) and/or user interfaces (1120), or to only control one of the two.

A second aspect of the invention relates to the use of the urinary measuring device according to any of the embodiments of the first aspect of the invention for urine analysis. Any of the urinary measuring devices disclosed with respect to FIGS. 1 to 11 can be used.

In a preferred embodiment of the second aspect of the invention, the analysis comprises a spectrophotometry measurement. Spectrometry, and specifically spectrophotometry, allows measuring, according to the Beer-Lambert law, the amount of a known chemical in a substance based on the light intensity when a light beam passes through a sample solution. This is because light is partially absorbed by the chemicals in the sample solution, and therefore the light intensity reveals the amount of said chemicals in the sample solution. In particular, the light received by the sensor from the UCU indicates an absorption spectrum which can be compared to the absorption spectrum of the components found in urine. In some embodiments, information from the sensor about the light received from the UCU is transmitted to a server comprising artificial intelligence software trained to detect one or more substances in a sample based on their absorption spectrum.

Therefore, given a urine sample, spectrophotometry allows the amount of different chemical substances in the sample to be measured by measuring the light intensity when light passes through the sample. The urinary measuring device according to any of the embodiments of the first aspect of the invention allows spectrophotometry to be performed by means of the optical system formed by a light emitter and a light sensor, in combination with the urine collection unit.

In a preferred embodiment of the second aspect of the invention, where the urinary measuring device comprises a plurality of electrodes, the analysis comprises measuring the electrical impedance between the electrodes. By applying a predetermined signal between two electrodes, the impedance of the electrodes can be determined depending on the signal received in one of the electrodes if the medium is conductive. Different media conduct electricity differently, so the impedance of one medium can be used to determine certain aspects of the medium. It can be used, for example, to differentiate between two solutions with different solute concentrations. It can also be used to differentiate between two different solutions.

Therefore, given a liquid sample, spectrophotometry allows measuring if the sample in the urinary receptacle is water or urine, as well as the concentration of the urine, by measuring the impedance of the medium between two electrodes. Concentration can be determined through artificial intelligence algorithms predicting impedance, or for example, the impedance can be compared with a lookup table to determine whether the sample is water or urine, and if the sample is urine, urine concentration (the relationship of impedance with urine concentration in the solution of the sample is known) and therefore urine density. In other embodiments, the impedance measurements are transmitted to a server comprising artificial intelligence software trained to determine urine concentration based on a measured impedance. The urinary measuring device according to any of the embodiments of the first aspect of the invention, wherein the urinary measuring device comprises a funnel and at least two electrodes, allows measuring the impedance of a solution by means of the electrodes arranged in the funnel.

A third aspect of the invention relates to a method for urine analysis comprising the following steps:
a) Receiving a urine sample in a container;
b) Emitting a light on the sample by means of a light emitter;
c) Receiving the light coming from the sample by means of a light sensor;
d) Transmitting data indicative of the light received by the sample to a processor configured to determine the presence of one or more substances in the sample based on the light received by the calculated sample; and
e) Receiving from the processor an indication of the presence of one or more substances in the sample.

In a preferred embodiment, the step of transmitting data indicative of the light received comprises the steps of calculating light absorption by the sample based on the light received and transmitting data indicative of light absorption to the processor which is configured to determine the presence of one or more substances in the sample based on light absorption by the sample.

The processor can be located in the device of the invention or can be a processor of an external device, such as a remote server.

In this method, it follows that the light emitter provides the light on the sample such that it is capable of interacting with said sample. Similarly, it follows that the sample allows the exit of the incident light after interacting with the sample and that the light sensor is arranged such that it receives the light coming from the sample. Light absorption is calculated based on the light received by means of the Beer-Lambert law. The absorbance of the solution with a wavelength λ can be obtained such that:

$${A}_{λ} = - {log}_{10} ( \frac{{I}_{1}}{{I}_{0}} ) .$$

where I₀ is the incoming light intensity and I₁ the outgoing light intensity.

Lastly, the presence of one or more substances in the sample can be derived from the absorbance for a known device. Given the distance I traveled by the light and the absorptivity of the body ε, the concentration of a substance in the medium can be obtained such that:

$$c = \frac{A}{l ε} .$$

Given several known substances in a sample, an equivalent analysis allows the concentration of each of said substances in the sample to be calculated. Furthermore, in some embodiments, the light emitter and the light sensor can emit and receive, respectively, light at different wavelengths. A plurality of wavelengths can be emitted and detected simultaneously or sequentially. In that sense, in an event of a sample with unknown substances, the type of substances therein can be derived, since different substances provide different colorations to the sample, and absorbance at different wavelengths varies. In this sense, some of the colorations of the sample comprised in the ultraviolet-visible (UV-vis) spectral range, which are known in the field of the art, are as follows:
Colorless samples are indicative of diuresis.
Yellowish samples are indicative of the presence of the urochrome pigment.
Pale yellowish samples are indicative of a good hydration. In combination with other factors, pale yellowish samples can indicate diabetes insipidus.
Whitish samples are indicative of lipiduria, chyluria, and urinary infections with neutrophils.
Pinkish samples are indicative of the presence of uric acid.
Reddish samples are indicative of the presence of red blood cells, (micro- or macrohematuria), which can in turn indicate the presence of kidney stones, urinary tract infections, and/or tumors.
Samples with brownish tones are indicative of the presence of waste from hemolysis, which can in turn indicate glomerulonephritis, malaria infection, reaction to transfusions, hemolysis-causing toxins (such as snake venom). These sample can also indicate processes resulting from excessive exercise, sepsis, metabolic disorders, medicaments, and toxins.
Orangey samples are indicative of the presence of secretions related to rifampin therapy. In children, orangey samples can be indicative of Lesch-Nyhan syndrome.
Bluish-greenish samples are indicative of the presence of bluish compounds mixed with urochrome. These compounds are derived from phenols. Furthermore, bluish-purplish samples can be indicative of propofol-related infusion syndrome in patients administered with propofol.
Samples with purplish tones are indicative of the presence of indirubin derived from catheter discoloration in patients with long-term catheterizations. These sample are also indicative of the presence of fine indigo blue crystals derived from bacterial oxidation of tryptophan in the digestive tract.
Greyish-blackish samples are indicative of the presence of pigments such as hemoglobin, melanin, porphyrin, or myoglobin, which mainly appear when urine is alkaline.

Therefore, in the embodiments in which the light emitter emits a plurality of light wavelengths such that the preceding colors can be distinguished by the device, the device can be configured to categorize the color of the sample according to the preceding categories and determine the presence of one or more possible substances or pathologies associated with the color.

These tones and other biochemical determinations observed between the UV-vis and infrared ranges can be measure with different wavelengths. For example, the urea in urine can be detected in an absorption spectrum with wavelengths comprised between 2000 and 2250 nm, creatinine with waves between 2100 and 2300 nm, and glucose with light waves between 1300 and 2500 nm. Hematocytes (hematuria) can be detected in an absorption spectrum with wavelengths comprised between 520 and 780 nm (red light). However, when a plurality of waves at different wavelengths is provided on the sample, the same effects can be achieved.

For example, the following combinations of wavelengths allow measuring:
Urea: 1400, 1500, 1700, 1800, 2100, 2200, 2300
Creatinine: 1400, 1600, 1700, 1800, 2100, 2200.
Sodium: 1400, 1600, 1700, 1800, 2100, 2200 nm

Similarly, combinations of wavelengths can be emitted to detect other substances in urine, for example, potassium (K⁺), calcium (Ca²⁺), magnesium (Mg²⁺), chloride (Cl⁻), hydrogen phosphate (HPO₄²⁻), and/or hydrogen carbonate (HCO₃⁻), bilirubin, nitrates, and ketones.

The use of certain wavelengths also allows measuring certain characteristics such as turbidity. Turbidity is derived from the presence of small, suspended particles that do not dissolved in the sample and is indicative of the presence of proteins in urine. It can be detected in an absorption spectrum with wavelengths comprised between 580 and 1000 nm for measuring turbidity. In urine samples, the value of turbidity is correlated with the amount of proteins dissolved in the sample, which means that protein concentration in the sample can be determined based on the measured turbidity.

It should be observed that determination of the concentration of a substance in a urine sample is not a clinical diagnosis, since other factors must be taken into account to determine the relevance and origin of said substance. However, it is a very important information source for the clinical setting or for another field of interest.

In an embodiment in which the container comprises an outlet opening, the emission of a light on the sample and the reception of the light coming from the same are started when the presence of a sample in the container is detected; and wherein the emission of the light on the sample is stopped and the reception thereof are stopped when the absence of a sample in the container is detected. It is observed that the presence and absence of a sample in the container can be detected by various sensors configured to detect the presence or absence of a sample in a container such as floats, measurements of an electromagnetic signal in the sample with electrodes, or any other means which allow detecting a volume in the container. For example, a float connected to a potentiometer could indicate the fluid level in the container. In a preferred embodiment, the presence and absence of the sample is detected by means of analyzing the electric current between a plurality of electrodes resulting from a voltage signal between a plurality of electrodes, wherein a current value above a threshold value between the electrodes indicates the presence of the sample, and a current value below a threshold value between the electrodes indicates the absence of the sample. In another embodiment, the presence and absence of the sample is detected by means of analyzing the electrical impedance measured between a plurality of electrodes, wherein an impedance magnitude value below a threshold value between the electrodes indicates the presence of the sample, and an impedance magnitude value above a threshold value between the electrodes indicates the absence of the sample.

In one embodiment, the method, after detecting the presence of a sample in the container, further comprises the following steps:
a) Measuring the impedance between the electrodes continuously until the absence of a sample in the container is detected;
b) Transmitting data indicative of the impedance of the sample to a processor configured to determine the concentration of the urine sample based on the measured impedance; and
c) Receiving from the processor an indication of the concentration of the sample.

The measurement of impedance between two electrodes allows measuring the concentration of the sample in which the electrodes are submerged. By means of the emission of waves for one frequency through a medium, it is possible obtain the impedance of the medium to the frequency. Different substances (the medium and the substances contained therein) have different impedance values, which allows obtaining the concentration of the sample. In one embodiment, the measured impedance is transmitted to an artificial intelligence algorithm which is trained to detect urine concentration. The artificial intelligence algorithm is trained using a data set of impedance values of urine samples having different urine concentration values.

In one embodiment, the method, after detecting the presence of a sample in the container, further comprises the following steps:
a) Starting a stopwatch when the presence of the sample in the container is detected;
b) Stopping the stopwatch when the absence of the sample in the container is detected;
c) Calculating the time elapsed in the stopwatch;
d) Transmitting data indicative of the time elapsed to a processor configured to determine the volume of the urine sample based on the time elapsed; and
e) Receiving from the processor an indication of the volume of the sample.

As explained in the first aspect of the invention, given a container having a known geometry with an outlet opening and a known outflow, it is possible to calculate the volume of passage through same based on the time it takes for the volume of sample to run through the container.

In one embodiment, the characteristics of the sample (urine density (mass per unit of volume), urine color, total urine volume, total urination time, mean urinary flow (volumetric flow rate), the presence and/or concentration of one or more substances in the urine, including electrolytes (for example, sodium (Na⁺), potassium (K⁺), calcium (Ca²⁺), magnesium (Mg²⁺), chloride (Cl⁻), hydrogen phosphate (HPO₄²⁻) and/or hydrogen carbonate (HCO₃⁻)), urea, creatinine, glucose, nitrate, ketones, proteinuria or hematuria, as well as the possibility of the user having one or more pathologies including dehydration, urinary tract infection, or kidney stones (urolithiasis), are determined by means of analyzing, in a computer or an external set of external computer programs, the measured associated values of the sample. Therefore, the presence of one or more substances in the sample is determined by means of analyzing light absorption in an external computer, and/or the concentration of one or more substances in the sample is determined by means of analyzing the electromagnetic signal between the electrodes submerged in the sample in an external computer, and/or the volume of the sample is determined by means of analyzing the time elapsed in the stopwatch in an external computer.

In a preferred embodiment of the third aspect of the invention, before receiving a urine sample, the user is identified through a user interface.

In a preferred embodiment of the third aspect of the invention, after discharging the cleaning liquid (for example, water), the method further comprises the following steps:
a) Discharging water or cleaning liquid when the absence of the sample in the container is detected;
b) Monitoring the presence of water in the container by means of the electrodes, by means of analyzing the electric current between the electrodes resulting from a voltage signal between a plurality of electrodes or by means of analyzing the electrical impedance measured between a plurality of electrodes; and
c) Determining that a new urine sample can be received when the presence of water is no longer detected by means of the electrodes, wherein a current value below a threshold value between the electrodes indicates the absence of water or an impedance magnitude value above a threshold value between the electrodes indicates the absence of water.

In one embodiment, the signal circulating between the electrodes may not be used continuously, but rather dichotomously, since analysis is not performed on the conductivity of the medium, but rather on the presence or absence thereof. In other embodiments, the signal may be used continuously.

In one embodiment, the electrodes are arranged at different heights, which allows determining the remaining container emptying time. In other embodiments, it furthermore allows knowing the sample volume in the container at all times. Another type of information derived from the arrangement at different heights can be obtained with this system.

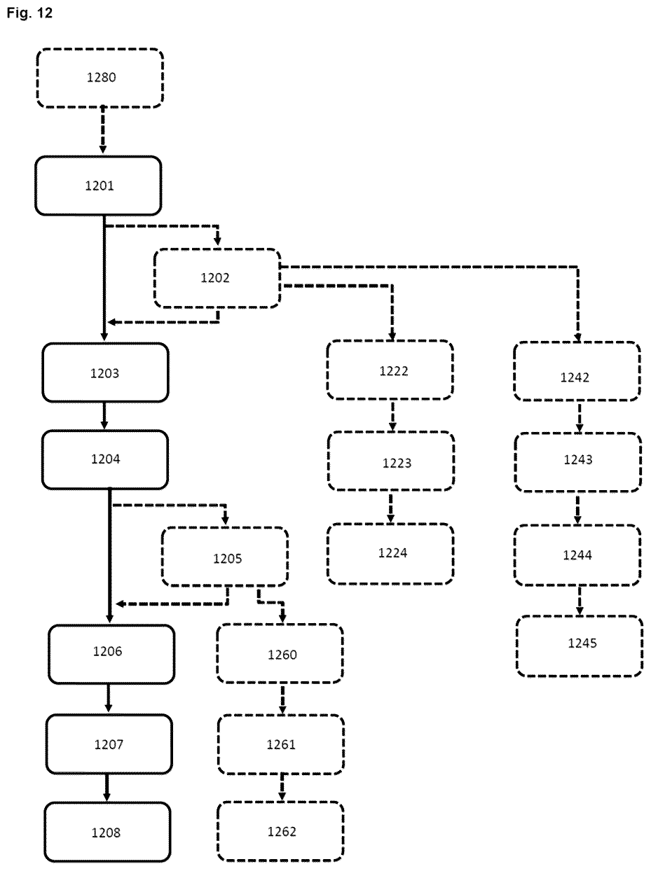

FIG. 12 shows a diagram of the urine analysis method according to the third aspect of the invention. The first step (1201) consists of receiving a urine sample, for example, in a sample container such as the UCUs herein disclosed. A light is then emitted on the sample by means of a light emitter (1203), and the light coming from the sample is received by means of a light sensor (1204). Once the light coming from the sample is received, light absorption by the sample is calculated based on the light received in comparison with a reference spectrum or with the light received during the prior discharge so as to normalize the measurements (1206). Data indicative of light absorption by the sample is then transmitted to a processor configured to determine the presence of one or more substances in the sample based on the calculated light absorption by the sample (1207). Lastly, an indication of the presence of one or more substances in the sample is received from the processor (1208).

Light absorption calculation (1206) can be performed immediately once the sensor (1204) starts receiving light, or after a certain time. Furthermore, the method of FIG. 12 contemplates a series of optional steps indicated by means of discontinuous arrows. First, it is possible to start emitting light on the sample by means of a light emitter (1203) and receiving light coming from the sample (1204) after detecting the presence of a sample in the container (1202), and to stop emitting light on the sample by means of a light emitter (1203) and receiving light coming from the sample (1204) after detecting the absence of a sample in the container (1205). The presence or absence of a sample in the container can be determined using any of the methods herein disclosed.

In some embodiments, the detection of the presence and absence of the sample in the container is performed by means of electrodes, although other means, such as infrared sensors or floats or ultrasound for measuring liquid height in the container, or a pressure sensor configured to detect the presence of liquid in the container are likewise object of this invention.

If an analysis is also to be performed on the concentration of the sample, once the electrodes arranged in the container detect the presence of the sample (1202), measurement of the signal circulating continuously between the electrodes will also be started, until the absence of a sample in the container (1222) is detected. Data indicative of the conductivity of the sample is the transmitted to a processor configured to determine the concentration of the urine sample based on the detected signal (1223), and lastly an indication of the concentration of the sample is received from the processor (1224).

If a volumetric analysis is also to be performed, after detecting the presence of a sample in the container, a stopwatch (1242) will be started. When the electrodes arranged in the funnel detect the absence of signal between the electrodes, the stopwatch (1243) is also stopped. Then, the time elapsed in the stopwatch (1244) is calculated and data indicative of the time elapsed is transmitted to a processor configured to determine the volume of the urine sample based on the time elapsed (1245). Lastly, an indication of the volume of the sample is received from the processor (1246).

Other optional steps involve determining when the urinary measuring device is ready to be used again. After detecting the absence of the sample in the container (1205), a valve is operated to discharge water into the sample container (1260). Furthermore, in some embodiments, there can be provided to the user a signal (for example, showing the user a warning on the user interface) which indicates that device recalibration is underway and new samples should not be added. The presence of water in the container of said tank (1261) is then monitored by means of the electrodes, wherein the signal flow between the electrodes indicates the presence of water. When the presence of water is no longer detected in the container by means of the electrodes, it is determined that a new sample can be received (1262). Furthermore, in some embodiments, there can be provided to the user, for example, by means of the user interface, a signal which indicates that the device is ready to be used again. In embodiments in which the user is identified, the results of the analysis by the device can be saved in a user profile saved in the memory, as well as in a set of computer programs in an external server.

Lastly, it is possible to establish a step prior to receiving the sample, wherein the user is identified through a user interface (1280).

All these methods of the third aspect of the invention can be implemented in a controller comprising a processor configured to execute a series of instructions to carry out the different method steps, and a memory unit configured to store the instructions.

Therefore, instructions can be defined so that the controller, once having received information from the electrodes that a sample has been received, starts the light emitter and the light sensor to begin a spectrophotometric analysis, and optionally starts a stopwatch. The analysis can be performed by a processor or data from the measurement can be transmitted to an external computer or server which analyzes the urine. Likewise, other instructions can be defined so that, once the controller having received information from the electrodes that a sample is no longer detected, the emission of light by the light emitter and the reception of light by the sensor are stopped, the stopwatch is optionally stopped, and a signal is optionally sent to a valve, preferably a solenoid valve, to discharge water.

Furthermore, other instructions can be defined so that, once the discharge of water is started, the results of the analysis are sent to user, and/or so that when the controller receives information from the electrodes that water is no longer detected, a signal which allows the user to know that he or she can perform the measurement is sent. This allows preventing the mixing of urine and water, properly cleaning the device, and calibrating the measuring systems for the next sample.

Instructions can also be defined so that the controller receives information identifying the user and associates the measurements of the sample with the user. All the samples can be sent through the controller to a set of external computer programs. This allows the viewing and treatment thereof by a third person, such as a specialist. The controller can also store the information of the device in its memory and receive instructions in order to send them to a computer program.

It is observed that any of the embodiments herein disclosed can be combined with any of the other embodiments.

In any of the embodiments herein disclosed, the light sensor may comprise one or more multichannel sensors configured to detect a plurality of wavelengths between 350 nm and 1000 nm. In one embodiment, the light sensor comprises at least eight channels, each detecting a different visible light wavelength (for example, 415 nm, 445 nm, 480 nm, 515 nm, 555 nm, 590 nm, 630 nm, and 680 nm) and optionally at least one channel in near infrared (for example, 910 nm). The light emitter is configured to emit light in a similar wavelength spectrum (for example, white light, or multiple light wavelengths simultaneously or sequentially).

FIG. 14 shows a computer program product 1400 for implementing the methods disclosed herein, which can be implemented in software, hardware, or a combination thereof. The module can be saved in a memory of the device (for example, in the casing for electronics or in the device comprising the user interface), or can be saved remotely, for example, in a remote server communicatively connected to the device. The computer program product 1400 is communicatively connected to the electronic components of the device. The computer program product 1400 can be configured to perform any of the steps of the method described in relation to FIG. 12.

The module comprises a spectrometry module 1435 which is configured to form a spectrophotometry analysis of the urine sample. The spectrometry module 1435 is configured to activate the light emitter of the device so that it emits light with predetermined characteristics (spectrum, intensity, etc.) on the support of the sample, and to receive data from the light sensor which is indicative of the light received by the light sensor in one or more wavelengths. The spectrometry module 1435 is configured to calculate the absorption of the sample in one or more wavelengths based on the data from the light sensor. In some embodiments, this is achieved by subtracting the data received from the sensor from a predetermined reference spectrum, saved in the memory, which is indicative of the data from the sensor expected for the one or more wavelengths when a sample is not present in the UCU. In some embodiments, this is achieved using the calibration module 1440 which is described in more detail below. The spectrometry module 1435 is configured to determine urine density (mass per unit of volume), urine color, total urine volume, total urination time, mean urinary flow (volumetric flow rate), the presence and/or concentration of one or more substances in the urine, including electrolytes (for example, sodium (Na⁺), potassium (K⁺), calcium (Ca²⁺), magnesium (Mg²⁺), chloride (Cl⁻), hydrogen phosphate (HPO₄²⁻) and/or hydrogen carbonate (HCO₃⁻)), urea, creatinine, glucose, nitrates, ketones, proteinuria or hematuria, as well as the possibility of the user having one or more pathologies including dehydration, urinary tract infection, or kidney stones (urolithiasis). In some embodiments, the color, turbidity, or the presence or concentration of one or more substances in the sample is determined by comparing the absorption values calculated for one or more wavelengths with the absorption values characteristic of the color and/or one or more substances and/or pathologies saved in the memory. The comparison can generate multiple candidates for the substances or pathologies for a specific calculated absorption spectrum. The candidates can be sent to a user interface, for example, to be shown to the user or to be saved in a user profile if the user has been identified, or the result can be sent to a device belonging to a third party (for example, the device belonging to the doctor) in order to request further investigation. In some embodiments, the spectrometry module 1435 can include artificial intelligence software which has been trained to detect the color of the sample and/or the turbidity and/or the presence or concentration of one or more substances and/or pathologies based on the absorption calculated. In the case of the embodiments in which the light sensor detects a plurality (for example, eight or more) of wavelengths, the artificial intelligence software can be trained to detect harmless substances such as medicaments, vitamins, and/or foods and beverages. This reduces the likelihood of a false positive result for a substance or pathology.

The artificial intelligence software is trained using training data obtained when executing the spectrometry module 1435 for training samples comprising colors, turbidities, and/or known amounts of target substances. Data from the sensors obtained for the training samples is provided to the artificial intelligence software as training data, together with a quantitative description of each training sample which describes the color, turbidity, and/or substances contained in each training sample and/or the pathologies indicated by the training samples. The artificial intelligence software is thereby trained to detect and distinguish the plurality of substances and pathologies.

In the embodiments in which the urinary measuring device comprises a sensor in the container configured to detect the presence of a sample in the received container of the UCU, the computer program product 1400 may comprise a sample detection module 1410 which is configured to receive data from the sensor in the container. The sample detection module 1410 can first turn all the other electronic components of the device on or can transition to a lower energy consumption mode. When the sensor in the container indicates to the sample detection module 1410 that there is a sample in the container, the sample detection module 1410 sends control signals to the other electronic components (for example, the optical system and the electrodes) in order to turn them on or put them in active mode. The sample detection module 1410 can cause one or more of the other modules of the computer program product 1400 to start. When the sensor in the container indicates to the sample detection module 1410 that there is no sample in the container, the sample detection module 1410 sends control signals to the other electronic components (for example, the optical system and the electrodes) in order to turn them off, or in a lower energy consumption mode. Accordingly, the sample detection module 1410 reduces energy consumption by selectively powering the components only when necessary. In the embodiments in which the sensor in the container comprises an impedance meter and a plurality of electrodes, the sample detection module 1410 can cause the impedance meter to emit a voltage signal continuously or intermittently and to measure the impedance. The sample detection module 1410 can compare the magnitude of the measured impedance with a predetermined threshold value saved in the memory, wherein if the measured impedance is below the threshold value, the sample detection module 1410 determines that there is a sample present in the container and the electronic components are turned on and several modules of the computer program product 1400 are activated. In the embodiments in which the sensor in the container comprises a voltage source between the electrodes and a current meter to measure the current between the electrodes, the sample detection module 1410 can cause the voltage source to emit a predetermined voltage signal and the current meter to measure the current. The sample detection module 1410 can compare the measured current with a predetermined threshold value saved in the memory, wherein if the measured current is above the threshold value, the sample detection module 1410 determines that there is a sample present in the container and the electronic components are turned on and several modules of the computer program product 1400 are activated. Alternatively or additionally, the electronic components of the device can be turned on or off manually through a user interface.

In some embodiments, the computer program product 1400 may comprise a washing module 1415. The washing module 1415 is configured to control a valve connected to a water source or a source for another cleaning solution for cleaning the device, and in particular the UCU. The water or the cleaning solution is preferably transparent to the wavelengths emitted by the optical system of the device in the embodiments in which the calibration module 1440 is present. When the sample detection module 1410 detect that there is no sample in the container, indicating that urination has ended, the sample detection module 1410 instructs the washing module 1415 to open the valve such that the UCU is washed. Alternatively or additionally, the washing module 1415 can be manually operated by a user through a user interface.

In some embodiments, the computer program product 1400 may further comprise a timing module 1420. When the sensor in the container indicates to the timing module 1420 that there is a sample present in the container, the timing module 1420 starts a stopwatch. When the sensor in the container indicates to the timing module 1420 that there is no sample in the container, the timing module 1420 stops the stopwatch. The timing module 1420 then calculates the total time elapsed which corresponds to the total urination time. The total time elapsed can be saved in a memory. The computer program product 1400 may further comprise a volume calculation module 1425 which calculates the total volume of the sample provided based on the calculated total urination time. This can be calculated by comparing total urination time with a lookup table saved in the memory or can be calculated using the equation which links total urination time with total volume for the given container shape. Calculated total urination time and/or total volume can be sent to the user interface to be presented to the user, or to be saved in a user profile, or the result can be sent to a device belonging to a third party (for example, a medical device).

In the embodiments in which the device comprises a plurality of electrodes in the container, the module 1400 may comprise a urinary concentration module 1430. The urinary concentration module 1430 is configured to control the voltage or current source and to receive measurement data from the impedance meter. The urinary concentration module 1430 can be configured to cause the voltage or current source to emit a predetermined AC or DC voltage or current constantly. In the embodiments in which the sample detection module 1410 is present, the urinary concentration module 1430 is activated when the sample detection module 1410 determines that there is a sample present and instructs the voltage or current source to emit a current or voltage. The current or voltage can be AC or DC and can be emitted continuously or intermittently. When an AC is emitted, the source can emit sequential signals of different frequencies. The urinary concentration module 1430 receives impedance measurements from the impedance meter indicating the impedance of the sample in the container. In some embodiments, the amplitude of the measured impedance is compared with a lookup table in the memory to determine the urine concentration of the sample. The impedance measurement value can be an average of a number of different impedance measurements so as to reduce the possibility of abnormal measurements. In other embodiments, the urinary concentration module 1430 comprises artificial intelligence software trained to determine urine concentration based on the magnitude of the impedance. The artificial intelligence is trained using a training data set of multiple urine samples with known concentrations and their corresponding measured impedances. In other embodiments, the urinary concentration module 1430 is comprised in the fusion module 1445 comprising artificial intelligence software trained to determine urine concentration based on the magnitude of the impedance and the absorption of the sample (both data can indicate urine concentration of urine). The artificial intelligence is trained using a training data set of multiple urine samples with known concentrations and their corresponding impedances and absorptions at several measured wavelengths. The calculated urine concentration value can be emitted to the user interface to be presented to the user, or can be saved in a user profile, or the result can be emitted to a device belonging to a third party (for example, the device belonging to the doctor). The urinary concentration module 1430 can furthermore compare the calculated concentration value with a plurality of predetermined ranges of threshold which indicate the hydration state of the user and emit the result. For example, if the concentration is within a range of threshold which indicates severe dehydration, the urinary concentration module 1430 emits a result which indicates that the user is severely dehydrated. For example, if the concentration is within a range of threshold which indicates a mild dehydration, the urinary concentration module 1430 emits a result which indicates that the user is slightly dehydrated. For example, if the concentration is within a range of threshold which indicates a suitable hydration, the urinary concentration module 1430 emits a result which indicates that the user is suitably hydrated. The result can be shown to the user through the user interface, and/or can be saved in a user profile, and/or can be sent to a device belonging to a third party.

In some embodiments, the computer program product 1400 comprises a calibration module 1440. The calibration module 1440 is configured to activate the light emitter of the device so that it emits light with predetermined characteristics (spectrum, intensity, etc.) on the support of the sample, and to receive data from the light sensor which is indicative of the light received by the light sensor in one or more wavelengths, after the device has been washed, for example by means of the activation of the washing module 1415 through manual activation or by the sample detection module 1410. A predetermined time after the washing module 1415 opens the valve, an indication is sent to the calibration module 1440 to start a calibration process. The calibration process comprises controlling the light emitter so that it emits the light with predetermined characteristics on the UCU, and so that it receives data from the light sensor which is indicative of the light received by the light sensor in one or more wavelengths. The data received is saved in the memory as the new reference spectrum. When the spectrometry module 1435 performs a new analysis, the new reference spectrum is subtracted from the data received from the sensor of the sample in order to calculate the absorption spectrum of the sample. The calibration module 1440 can be configured to obtain a new reference spectrum, as indicted above, every time the device is washed, or after a predetermined number of washed (such as every 2, 5, 10, or more washes). Therefore, the calibration module 1440 takes into account gradual deterioration or discoloration of the support of the sample, and/or the light signal emitted by the optical system, such that the reference spectrum reflects the actual expected signal received by the sensor if there is no absorbent sample. By dynamically updating the reference spectrum used to calculate the absorption spectrum, the device is capable of calculating an accurate absorption spectrum even when, for example, the UCU deteriorates or becomes discolored over time, which in turn affects the light spectrum detected by the light sensor.

In some embodiments, the computer program product 1400 comprises a fusion module 1445. The fusion module 1445 combines data from two or more of the timing module 1420, volume calculation module 1425, urinary concentration module 1435, and spectrometry module 1435 and determines urine concentration. In some embodiments, the computer program product 1400 comprises a summary module 1450 which is configured to receive the results from any of the other modules and provide a summary which describes in detail the detected characteristics of the sample (for example, the presence of one or more substances, possibility or one or more pathologies, hydration level). The summary module 1450 can furthermore shows the possible actions to be taken based on the detected characteristics, which are assigned to the detected characteristics of the sample in a memory. The results can be sent to a user interface, for example, in order to show them to the user or to save them in a user profile if the user has been identified, or the result can be sent to a device belonging to a third party (for example, a medical device).

### CLAUSES

1. A urinary measuring device for taking and measuring urine samples, configured to be arranged in a urinary receptacle; characterized in that it comprises:
a. A funnel for sample collection, comprising a main opening;
b. A urine collection unit connected to the opening for receiving the sample from the funnel;
c. A casing for electronics positioned close to the urine collection unit, the casing for electronics containing a light emitter arranged for emitting light on the urine collection unit and a light sensor arranged for measuring the light received from the urine collection unit.

2. The urinary measuring device according to clause 1, characterized in that the funnel comprises a filter that at least partially covers the opening configured to prevent direct urination through the opening.

3. The urinary measuring device according to any of clauses 1 or 2, characterized in that the funnel further comprises a gasket in a part of the urinary measuring device, the gasket configured to contact the urinary receptacle for fluidically sealing a space between the urinary measuring device and the urinary receptacle.

4. The urinary measuring device according to any of clauses 1 to 3, characterized in that the funnel further comprises an overflow opening configured to inhibit the sample from overflowing from the funnel, and a conduit configured to receive fluid from the overflow opening, the conduit bypassing the urine collection unit.

5. The urinary measuring device according to clause 4, characterized in that the funnel further comprises a lid covering the overflow opening configured to prevent the sample from flowing directly through the overflow opening.

6. The urinary measuring device according to any of clauses 1 to 5, characterized in that the light emitter and the light sensor are located in the casing for electronics for emitting and receiving light with respect to the urine collection unit at an angle comprised between 120° and 180°.

7. The urinary measuring device according to any of clauses 1 to 6, characterized in that the casing for electronics comprises a main body, a cover, and a gasket configured to fluidically seal the interface between the main body and the cover.

8. The urinary measuring device according to any of clauses 1 to 7, characterized in that the casing for electronics comprises a transparent protector arranged for protecting the light emitter and the light sensor against the sample splashing from the urine collection unit.

9. The urinary measuring device according to any of clauses 1 to 8, characterized in that the urine collection unit comprises a surface arranged for maximizing the reflection of an incident light and which in turn comprises a material selected from one of the materials from the following list: ceramic, crystal, glass, porcelain, metal, silicone, wood, stones such as marble, polymers such as POM or photosensitive resin, as well as a derivative of these materials.

10. The urinary measuring device according to any of clauses 1 to 9, characterized in that the device further comprises:
d. A container for collecting fluid overflowing from the urine collection unit, comprising an outlet opening; and
e. A plurality of electrodes extending inside the container by means of arms, and optionally:
   a voltage source connected between the electrodes configured to apply a voltage signal between the electrodes and a current meter configured to measure the current between the electrodes resulting from a voltage signal applied by the voltage source; or
   an impedance meter connected to the electrodes, the impedance meter configured to measure the impedance of the fluid present in the container between the electrodes.

11. The urinary measuring device according to clause 10, characterized in that the outlet opening of the container is configured to allow an outflow smaller than that of the main opening of the funnel.

12. The urinary measuring device according to any of clauses 10 or 12, characterized in that the container further comprises a side outlet opening, arranged at a greater height than that of the outlet opening and than that of the plurality of electrodes.

13. The urinary measuring device according to any of clauses 10 to 12, characterized by electric cables extending from the arms to the casing for electronics through one or more gaskets, in order to form an electrical connection between the electrodes and the electronics in the casing for electronics.

14. The urinary measuring device according to any of clauses 1 to 13, characterized in that the funnel comprises a handle configured to facilitate the extraction of the urinary measuring device by means of an extraction device.

15. The urinary measuring device according to any of clauses 1 to 14, characterized in that the casing for electronics further comprises a controller configured to control the light emitter, the light sensor, and optionally the impedance meter, and wherein the controller is made up of a processor configured to process information from the light emitter, the light sensor, and optionally from the impedance meter.

16. The urinary measuring device according to any of clauses 1 to 15, characterized in that the urinary measuring device further comprises a valve configured to control the outflow of water from a water source to the urinary receptacle and wherein the device is configured to control the valve.

17. The urinary measuring device according to clause 16, characterized in that the urinary measuring device is configured to open the valve when it detects that sample measurement has ended.

18. The urinary measuring device according to any of clauses 1 to 17, characterized in that the urinary measuring device further comprises a user interface configured to receive identification or anonymous information of a user and to receive information about the urinary sample of the user and to show information about the condition of the sample to the user.

19. Use of the urinary measuring device according to any of clauses 1 to 18 for urine analysis.

20. Use of the urinary measuring device according to clause 19, characterized in that the analysis comprises a spectrophotometric measurement.

21. Use of the urinary measuring device according to clauses 19 or 20, characterized in that the urinary measuring device comprises a plurality of electrodes and an impedance meter and wherein the analysis comprises measuring the electrical impedance between the electrodes.

22. A method for urine analysis, comprising the following steps:
a) Receiving a urine sample in a container directly from a user, with the container being positioned in a urinary receptacle;
b) Emitting a light on the sample by means of a light emitter;
c) Receiving the light coming from the sample by means of a light sensor;
d) Transmitting data indicative of the light received by the sample to a processor configured to determine the presence of one or more substances in the sample based on the light received by the sample; and
e) Receiving from the processor an indication of the presence of one or more substances in the sample.

23. The method according to clause 22, characterized in that the step of transmitting data indicative of the light received comprises the steps of calculating light absorption or reflection by the sample based on the light received and transmitting data indicative of light absorption to the processor which is configured to determine the presence of one or more substances in the sample based on light absorption by the sample.

24. The method according to clause 22 or 23, characterized in that the emission of a light on the sample and the reception of the light coming from the sample start when the presence of a sample in the container is detected; and wherein the emission of light on the sample and the reception of light are stopped when the absence of a sample in the container is detected.

25. The method according to clause 24, characterized in that:
the presence and absence of the sample is detected by means of analyzing the electric current between a plurality of electrodes resulting from a voltage signal between a plurality of electrodes, wherein a current value above a threshold value between the electrodes indicates the presence of the sample, and a current value below a threshold value between the electrodes indicates the absence of the sample; or
the presence and absence of the sample is detected by means of analyzing the electrical impedance measured between a plurality of electrodes, wherein an impedance magnitude value below a threshold value between the electrodes indicates the presence of the sample, and an impedance magnitude value above a threshold value between the electrodes indicates the absence of the sample.

26. The method according to clause 25, characterized in that, after detecting the presence of a sample in the container, the method comprises the following steps:
a) Measuring the impedance between the electrodes continuously until the absence of a sample in the container is detected;
b) Transmitting data indicative of the conductivity of the sample to a processor configured to determine the concentration of the urine sample based on the measured impedance signal; and
c) Receiving from the processor an indication of the concentration of the sample.

27. The method according to any of clauses 25 or 26, characterized in that, after detecting the presence of a sample in the container, the method further comprises the following steps:
a) Starting a stopwatch when the presence of the sample in the container is detected;
b) Stopping the stopwatch when the absence of the sample in the container is detected;
c) Calculating the time elapsed in the stopwatch;
d) Transmitting data indicative of the time elapsed to a processor configured to determine the volume of the urine sample based on the time elapsed; and
e) Receiving from the processor an indication of the volume of the sample.

28. The method according to any of clauses 22 to 27, characterized in that one or more of the following characteristics of the sample are determined by means of analyzing the measured associated values of the sample in an algorithm performed by an external computer: urine density (mass per unit of volume), urine color, total urine volume, total urination time, mean urinary flow (volumetric flow rate), the presence and/or concentration of one or more substances in the urine, the possibility of the user having one or more pathologies.

29. The method according to any of clauses 22 to 28, characterized in that before receiving a urine sample, the user is identified through a user interface.

30. The method according to any of clauses 24 to 29, characterized in that it further comprises the following steps:
a) Discharging water from a water source when the absence of the sample in the container is detected;
b) Monitoring the presence of water in the container by means of the electrodes, by means of analyzing the electric current between the electrodes resulting from a voltage signal between a plurality of electrodes or by means of analyzing the electrical impedance measured between a plurality of electrodes; and
c) Determining that a new urine sample can be received when the presence of water is no longer detected by means of the electrodes, wherein a current value below a threshold value between the electrodes indicates the absence of water or an impedance magnitude value above a threshold value between the electrodes indicates the absence of water.

30. A computer program, connected with a container positioned in a urinary receptacle, comprising computer-readable instructions which, when being executed by a processor, cause the processor to execute a method according to the following steps:
a) Emitting a light by means of a light emitter on a urine sample from a user contained in a container, with the container being positioned in a urinary receptacle;
b) Receiving the light coming from the sample by means of a light sensor;
c) Transmitting data indicative of the light received by the sample to a processor configured to determine the presence of one or more substances in the sample based on the light received by the sample; and
d) Receiving from the processor an indication of the presence of one or more substances in the sample.

1. A urinary measuring device for taking and measuring urine samples, configured to be arranged in a urinary receptacle, the urinary measuring device comprising:
a. a funnel for sample collection, comprising a main opening:
b. a urine collection unit connected to the opening for receiving the sample from the funnel and comprising a light reflective surface;
c. a casing for electronics positioned close to the urine collection unit, the casing for electronics containing a light emitter arranged for emitting light on the urine collection unit and a light sensor arranged for measuring the light received by reflection from the urine collection-unit unit;
d. a container for collecting fluid overflowing from the urine collection unit, comprising an outlet opening, wherein the outlet opening of the container is configured to allow an outflow smaller than that of the main opening of the funnel, and
e. plurality of electrodes extending inside the container by means of arms.

2. The urinary measuring device according to claim 1, wherein the funnel comprises a filter that at least partially covers the main opening configured to prevent direct urination through the main opening.

3. The urinary measuring device according to claim 1, wherein the light emitter and the sensor are located in the casing for electronics for emitting and receiving light with respect to the urine collection unit such that the planes perpendicular to the emission and measurement directions form at an angle comprised between 120° and 180°.

4. The urinary measuring device according to claim 1, wherein the casing for electronics comprises a main body, a cover, and a gasket configured to fluidically seal the interface between the main body and the cover.

5. The urinary measuring device according to claim 1, wherein the casing for electronics comprises a transparent protector arranged for protecting the light emitter and the sensor against the sample splashing from the urine collection unit.

6. The urinary measuring device according to claim 1, wherein the urine collection unit comprises a surface arranged for maximizing the reflection of an incident light comprising a material selected from one of: ceramic, crystal, glass, porcelain, metal, silicone, wood, stones, polymers, as well as a derivative of these materials.

7. The urinary measuring device according to claim 1, characterized in that wherein the device further comprises:
a voltage source connected between the electrodes configured to apply a voltage signal between the electrodes and a current meter configured to measure the current between the electrodes resulting from a voltage signal applied by the voltage source; or
an impedance meter connected to the electrodes, the impedance meter configured to measure the impedance of the fluid present in the container between the electrodes.

8. (canceled)

9. The urinary measuring device according to claim 1, wherein the container further comprises a side outlet opening, arranged at a greater height than that of the outlet opening and than that of the plurality of electrodes.

10. The urinary measuring device according to claim 1, wherein electric cables extend from the arms to the casing for electronics through one or more gaskets, such that an electrical connection is formed between the electrodes and the electronics in the casing for electronics.

11. The urinary measuring device according to claim 1, wherein the funnel comprises a handle configured to facilitate the extraction of the urinary measuring device by way of an extraction device.

12. The urinary measuring device according to claim 1, wherein the casing for electronics further comprises a controller configured to control the light emitter and the light sensor, and wherein the controller comprises a processor configured to process information from the light emitter and the light sensor.

13. The urinary measuring device according to claim 1, wherein the urinary measuring device further comprises a valve configured to control the outflow of water from a water source to the urinary receptacle and wherein the device is configured to control the valve.

14. The urinary measuring device according to claim 13, wherein the urinary measuring device is configured to open the valve when it detects that sample measurement has ended.

15. The urinary measuring device according to claim 1, wherein the urinary measuring device further comprises a user interface configured to receive identification or anonymous information of a user and to receive information about the urinary sample of the user and to show information about the condition of the sample to the user.

16. The urinary measuring device according to claim 1, wherein the plurality of electrodes are configured to control the light emitter and light sensor.

17. The urinary measuring device according to claim 12, wherein the controller is configured to control the impedance meter, and wherein the processor is configured to process information from the impedance meter.
